(19) United States
(12) Patent Application Publication
(10) Pub. No.: US 2024/0191181 A1
(43) Pub. Date: Jun. 13, 2024

(54) METHODS AND SYSTEMS FOR NON-PLANAR FREEFORM FUSED DEPOSITION MANUFACTURING

(71) Applicant: CORNELL UNIVERSITY, Ithaca, NY (US)

(72) Inventors: Benjamin J. ALBERT, Ithaca, NY (US); Jonathan BUTCHER, Ithaca, NY (US)

(21) Appl. No.: 18/554,578

(22) PCT Filed: Apr. 8, 2022
(86) PCT No.: PCT/US2022/024062

§ 371 Date: Oct. 9, 2023

Related U.S. Application Data
(60) Provisional application No. 63/172,395, filed on Apr. 8, 2021.

(51) Int. Cl. C12N 5/00; B29C 64/118; B29C 64/336; B29C 64/386; B29K 105/00; B29L 31/00; B33Y 10/00; B33Y 50/00; B33Y 80/00

(52) U.S. Cl. CPC C12N 5/0062; B29C 64/118; B29C 64/336; B29C 64/386; B33Y 10/00; B33Y 50/00; B33Y 80/00; B29K 2005/00; B29K 2105/0061; B29L 2031/7532; C12N 2513/00; C12N 2533/54; C12N 2533/74; C12N 2537/10

(57) ABSTRACT

The present disclosure relates to a method of fabricating an article. This method involves obtaining a non-planar surface model of an article to be fabricated; creating X and Y coordinates of the surface to create an X-Y plane; creating planes orthogonal to the X-Y plane to obtain lines of intersection between the surface and said planes; correlating coordinates of intersecting lines to create non-planar print paths; and printing along the print paths to fabricate the article. Also disclosed are a system and a computer program product that functions to control a system to carry out the methods of the present disclosure.

This application claims the priority benefit of U.S. Provisional Patent Application Ser. No. 63/172,395, filed Apr. 8, 2021, which is hereby incorporated by reference in its entirety.

This invention was made with government support under 81023/A001 awarded by National Science Foundation. The government has certain rights in the invention.

### FIELD OF THE INVENTION

The present disclosure relates to methods and systems for non-planar freeform fused deposition manufacturing.

### BACKGROUND OF THE INVENTION

The introduction of 3D printing methods into the field of tissue engineering has expanded the possibilities of tissue manufacturing (Kang et al., “A 3D Bioprinting System to Produce Human-scale Tissue Constructs with Structural Integrity,” Nature Biotechnology 34(3):312-319 (2016); Kolesky et al., “3D Bioprinting of Vascularized, Heterogeneous Cell-laden Tissue Constructs,” Advanced Materials 26(19):3124-3130 (2014); Murphy et al., “3D Bioprinting of Tissues and Organs,” Nature Biotechnology 32(8): 773-785 (2014); Ozbolat & Hospodiuk, “Current Advances and Future Perspectives in Extrusion-based Bioprinting,” Biomaterials 76:321-343 (2016)). The increase in 3D bioprintable materials and methods has led to rapid advances in the field. However, there are still many hurdles to overcome for many clinical applications of bioprinting (Sun et al., “The Bioprinting Roadmap,” In Biofabrication 12(2) (2020)). Manufacturing tissues out of cellularly viable materials has been difficult because most biomaterials require an external factor to solidify the material to maintain its manufactured shape (Panwar & Tan, “Current Status of Bioinks for Micro-extrusion-based 3D Bioprinting,” Molecules 21(6) (2016)). Extrusion printing methods have often been used for bioprinting because of the wide range of materials that can be extruded through this deposition method (Lim et al., “Fundamentals and Applications of Photo-Cross-Linking in Bioprinting,” In Chemical Reviews 120(19): 10662-10694 (2020); Ouyang, “Pushing the Rheological and Mechanical Boundaries of Extrusion-based 3D Bioprinting,” Trends in Biotechnology 1-12 (2020)). This printing process requires thermal, chemical, UV crosslinking, or other methods to solidify the extruded material to maintain the desired structure (Murphy et al., “3D Bioprinting of Tissues and Organs,” Nature Biotechnology 32(8): 773-785 (2014); Ozbolat & Hospodiuk, “Current Advances and Future Perspectives in Extrusion-based Bioprinting,” Biomaterials 76:321-343 (2016)). When printing in open air, these materials may collapse or deform before the solidification can take effect (Lim et al., “New Visible-Light Photoinitiating System for Improved Print Fidelity in Gelatin-Based Bioinks,” ACS Biomaterials Science and Engineering 2(10): 1752-1762 (2016); Ouyang et al., “A Generalizable Strategy for the 3D Bioprinting of Hydrogels from Nonviscous Photo-crosslinkable Inks,” Advanced Materials 29(8) (2017)).

Recent advances in embedded printing have increased the resolution capability of extrusion bioprinting by holding the deposited material in place to allow for more time to solidify (Bhattacharjee et al., “Writing in the Granular Gel Medium,” Science Advances 1(8):4-10 (2015); Lee et al., “3D Bioprinting of Collagen to Rebuild Components of the Human Heart,” Science 365(6452):482-487 (2019); Ning et al., “Embedded 3D Bioprinting of Gelatin Methacryloyl-Based Constructs with Highly Tunable Structural Fidelity,” ACS Applied Materials & Interfaces 12(40):44563-44577 (2020)). These methods rely on a secondary bath of material that the biomaterial is extruded into. The rheological properties of the bath material hold the printed material in place relative to where it was deposited. This method has allowed for creation of complex vessels, heart valves, and additional structures that have been difficult to replicate with open air extrusion bioprinting (Lee et al., “3D Bioprinting of Collagen to Rebuild Components of the Human Heart,” Science 365(6452):482-487 (2019); Skylar-Scott et al., “Biomanufacturing of Organ-specific Tissues with High Cellular Density and Embedded Vascular Channels,” Science Advances 5(9) (2019)). The support of the bath means that the printed material is unaffected by gravity, meaning that the printing direction is only constrained by the capabilities of the printer (Shiwarski et al., “Emergence of FRESH 3D Printing as a Platform for Advanced Tissue Biofabrication,” APL Bioengineering 010904 (February) (2021)).

Traditional print slicing software for 3-axis printers is limited to printing a complete layer in the X-Y plane and then translation in the Z axis to start the next layer. Geometries that are discontinuous in any particular X-Y slice require travel moves between extrusion sites that may blend and distort the deposited material. A similar effect may be seen when printing with multiple materials in embedded printing methods. Extruder changes require removal of the current nozzle from the bath and insertion of a second nozzle. These travel moves may additionally disrupt the deposited material either through the nozzle itself or material oozing during travel moves.

Planar slicing can recreate many structures with embedded printing; however, the material deposition is limited to a single X-Y plane at any given time. Many tissues are composed of organic curves that may not be conducive to being sliced in this way. Tissues contain multiple domains of materials within a curved structure. In addition, these materials often align to create anisotropic properties that can be replicated through utilizing the print nozzle direction (Moncal et al., “Thermally-controlled Extrusion-based Bioprinting of Collagen,” Journal of Materials Science: Materials in Medicine 30(5) (2019); Prendergast et al., “A Biofabrication Method to Align Cells Within Bioprinted Photocrosslinkable and Cell-degradable Hydrogel Constructs via Embedded Fibers,” Biofabrication 0-23 (2021)). Controlling print curvature to match the natural curvature of a tissue may allow for control over material deposition and mechanical properties.

Recent studies using thermoplastics to create thin curved materials with a preferred print path direction have demonstrated non-planar printing, where the print path direction is not constrained to the X-Y plane (Shembekar et al., “Generating Robot Trajectories for Conformal Three-dimensional Printing Using Nonplanar Layers,” Journal of Computing and Information Science in Engineering 19(3): 1-13 (2019)). However, these printed thermoplastic materials still rely on a static pre-built support for the print to be deposited onto. Embedded printing of biomaterials allows for similar strategies to be implemented for bioprinting applications without the need for separate support structures.

The present disclosure is directed to overcoming these and other deficiencies in the art.

### SUMMARY OF THE INVENTION

One aspect of the present disclosure relates to a method of fabricating an article. This method involves obtaining a non-planar surface model of an article to be fabricated; creating X and Y coordinates of the surface to create an X-Y plane; creating planes orthogonal to the X-Y plane to obtain lines of intersection between the surface and said planes; correlating coordinates of intersecting lines to create non-planar print paths; and printing along the print paths to fabricate the article.

The present disclosure relates to a new method of slicing 3D bioprint models for non-planar movement to create a variety of structures applicable to tissue engineering. In some embodiments, the process is repeated with several different biomaterials and embedded printing techniques in a range of applications. The printing process of the present disclosure demonstrates the ability to tune mechanical properties and create complex materials using traditional bioinks and other suitable compositions.

### BRIEF DESCRIPTION OF THE DRAWINGS

FIGS. 1A-D illustrate one embodiment of a method for fabricating an article. In FIG. 1A, a digital three-dimensional model having a desired geometry is imported as an STL file. In FIG. 1B, paths are generated using planes that intersect with the model in either the X or the Y direction. Layers are repeated to generate the desired final thickness. In FIG. 1C, the print GCODE is loaded to the bioprinter and the print is created in an embedded print bath. In FIG. 1D, the print is then washed of the bath and the result is a solid print with the curvature of the initial model.

FIG. 2 is a flowchart of one embodiments of an algorithm used to instruct a bioprinter in methods of fabricating an article disclosed herein.

FIGS. 3A-H are a series of images showing steps involved in generating Gcode files for non-planar prints according to embodiments of a method of fabricating an article described herein. In step 1 (FIG. 3A), a surface model of a three-dimensional article to be fabricated is provided. In step 2 (FIG. 3B), a silhouette of intersecting X-Z and Y-Z planes is generated. In step 3 (FIGS. 3C-D), paths at plane-surface intersections are mapped. In step 4 (FIGS. 3E-F), nearest neighbor paths are connected. In step 4 (FIG. 3G), X paths and Y paths are combined. In step 5 (FIG. 3H), alternating layers are repeated to achieve the desired thickness.

FIGS. 4A-C show that print path consistency depends on print angle. FIG. 4A is a photograph showing representative print paths of 45 degree upward)(+45° printing and 45 degree downward)(−45° printing. FIG. 4B is a photograph also showing print path diameter with respect to print angle. FIG. 4C shows the diameter of extruded material with respect to angle of nozzle movement where 0° is horizontal.

FIGS. 5A-E show dual material planar and non-planar printing of a layered curve. FIG. 5A shows a model of alternating layers with an “M”-shaped curve. FIG. 5B is a representation of the non-planar print paths. FIG. 5C is a representation of the planar print paths. FIG. 5D shows a final print with non-planar slicing. FIG. 5E shows a final print with planar slicing.

FIGS. 6A-F show the impact of non-planar corrugation on print mechanics. FIG. 6A is a perspective view of a 3D model and printed corrugation. FIG. 6B shows a side profile of the four amplitude profiles ranging from flat to 45 degree angles. FIG. 6C is a graph showing representative stress-strain relationship of when stretched to failure. FIG. 6D is a graph showing modulus at 10% strain. FIG. 6E is a graph showing maximum modulus. FIG. 6F is a graph showing ultimate tensile strength.

FIGS. 7A-F show the impact of corrugation curvature on print mechanics. FIG. 7A is a perspective view of a 3D model and printed corrugation. FIG. 7B is a side view of four different fillet profiles ranging from no fillet to a 2 mm radius of curvature. FIG. 7C is a graph showing representative stress-strain relationship of when stretched to failure. FIG. 7D is a graph showing modulus at 10% strain. FIG. 7E is a graph showing maximum modulus. FIG. 7F is a graph showing ultimate tensile strength.

FIGS. 8A-E show that size and frequency of corrugation modifies stretch response in low strain regions. In FIG. 8A, prints were designed with paths aligned either parallel or perpendicular to the direction of stretch. FIG. 8B is a graph showing representative stress-strain relationship of when stretched to failure. FIG. 8C is a graph showing modulus at 10% strain. FIG. 8D is a graph showing maximum modulus. FIG. 8E is a graph showing ultimate tensile strength.

FIGS. 9A-E show varying print path direction across six layers of the print and the effect on stiffness and strength of final print. FIG. 9A is a schematic illustration showing that prints were designed with paths aligned either parallel or perpendicular to the direction of stretch. FIG. 9B is a graph showing a representative stress-strain relationship of when stretched to failure. FIG. 9C is a graph showing modulus at 10% strain. FIG. 9D is a graph showing maximum modulus. FIG. 9E is a graph showing ultimate tensile strength.

FIGS. 10A-E show non-planar printing of auxetic geometries. FIG. 10A is a perspective view of an example model of the Miura-Ori folded structure. FIG. 10B is a photograph showing a printed sample generated from the Miura-Ori model. FIG. 10C is a photograph showing flat and Miura-Ori with 6 mm depth of folds unstretched (left) and stretched to 20% strain (right). FIG. 10D is a graph showing representative stretch and release curves of flat and Miura-Ori samples with various depths of folded features. FIG. 10E is a graph showing Poisson ratio of flat and Miura-Ori samples.

### DETAILED DESCRIPTION OF THE INVENTION

The present disclosure relates to methods of fabricating non-planar objects, specifically methods and systems for non-planar freeform fused deposition manufacturing.

One aspect of the present disclosure relates to a method of fabricating an article. This method involves obtaining a non-planar surface model of an article to be fabricated; creating X and Y coordinates of the surface to create an X-Y plane; creating planes orthogonal to the X-Y plane to obtain lines of intersection between the surface and said planes; correlating coordinates of intersecting lines to create non-planar print paths; and printing along the print paths to fabricate the article.

In the present disclosure, it is noted that traditional 3D additive manufacturing methods that slice the representation of the 3D object into parallel planes create slices that are not intrinsic to the original 3D object representation, and may result in inferior mechanical strength, material properties, surfaces finishes, and the like, that would otherwise be possible.

The methods disclosed herein and products and systems used to carry out such methods, including, e.g., code executable by processor(s), create intersecting planes in X-Z and Y-Z axes, and thereby address technical challenges that are not solved by other methods. Although other attempts have been made to print 3D objects with non-planar surfaces without slicing, or using other modes of slicing, such methods do not provide complete coverage of the volume of the 3D object and leave unsolved technical challenges that are addressed by the methods and/or systems described herein.

For example, Mueller et al., “Wireprint: 3d Printed Previews for Fast Prototyping,” In Proceedings of the 27th Annual ACM Symposium on User Interface Software and Technology, 273-280 (2014), which is hereby incorporated by reference in its entirety, describes how a wireframe on the boundary of an object can be printed directly without slicing to quickly create a visual representation of the object. However, only a wireframe representation of the boundary is created by this method and materials are required to be self-supporting.

Bourell et al., “A Layerless Additive Manufacturing Process Based on cnc Accumulation,” Rapid Prototyping Journal 17(3):218-227 (2011) and Pan et al., “Multitool and Multi-axis Computer Numerically Controlled Accumulation for Fabricating Conformal Features on Curved Surfaces,” Journal of Manufacturing Science and Engineering 136(3): 031007 (2014), which are hereby incorporated by reference in their entirety, appear to described how manually planned print-paths are used to create objects without slicing. The method relies on manual definitions by the user, and the focus appears to be on implementing and testing the hardware needed to accomplish the prints.

In Huang et al., “Curved Layer Adaptive Slicing (clas) for Fused Deposition Modelling,” Rapid Prototyping Journal 21(4) (2015), which is hereby incorporated by reference in its entirety, non-planar slices are created and used to print special geometries. However, the objects are limited to geometries that can be expressed as offsets of polygonal faces (e.g., as described with reference to Qu and Stucker, “A 3d Surface Offset Method for stl-format Models,” Rapid Prototyping Journal 9(3): 133-141 (2003), which is hereby incorporated by reference in its entirety, starting from the top (facing up) polygonal surface of the object. For example a sphere cannot be printed since the bottom is not an offset of the top, while an arch shaped bridge structure can be printed. The mentioned future work seeks to handle more complex geometries by subdividing an object. However, the division is into two groups: parts that can be printed using non-planar slices using the described method, and complex parts that are still printed using traditional slicing methods.

Gao et al., “Revomaker: Enabling Multi-directional and Functionally-embedded 3d Printing Using a Rotational Cuboidal Platform,” In Proceedings of the 28th Annual ACM Symposium on User Interface Software & Technology, 437-446 (2015), which is hereby incorporated by reference in its entirety, appears to introduce an additional degree of freedom to 3D printing, and allows printing around a cuboid (box) object and creating slices in six different directions corresponding to the facets of the cuboid. However, the resulting object is, in essence, made of six traditionally sliced (and manufactured) objects fused together.

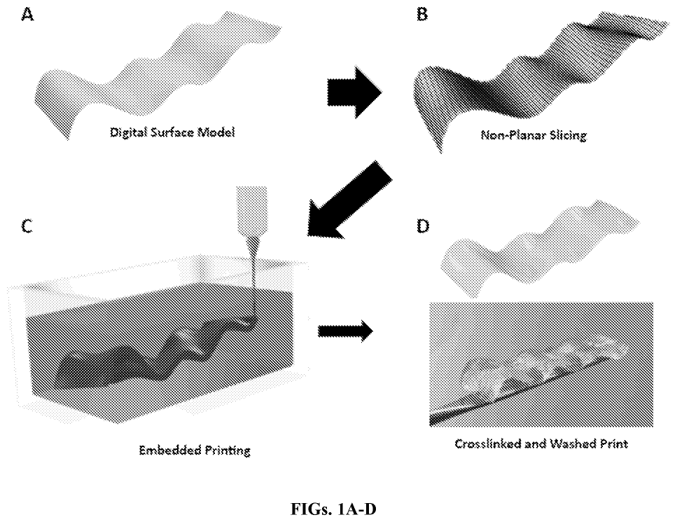

In contrast, in some embodiments, the present disclosure relates to a method of fabricating an article as shown in the schematic illustration of FIGS. 1A-D. Specifically, a non-planar surface of an article to be fabricated is obtained and/or provided. In some embodiments, the non-planar surface of an article to be fabricated (Digital Surface Model) is obtained in a digital format, as illustrated in FIG. 1A, such as computer-aided design (“CAD”) data. Whatever the geometry of the Digital Surface Model, a design algorithm is then used to define the non-planar surface of the article to be manufactured. In some embodiments, the algorithm creates X-Z and Y-Z planes to obtain lines of intersection between the surface and these planes, and Z values of the surface and plane intersections are correlated to the X and Y coordinates to create non-planar print paths, as illustrated in FIG. 1B. The design algorithm instructs a 3D printer to then print the article. In some embodiments, the article is printed using embedded printing, as illustrated in FIG. 1C. Embedded printing involves printing the article in a support medium. Once the article is printed, it is cross-linked or gelled and then washed, as illustrated in FIG. 1D.

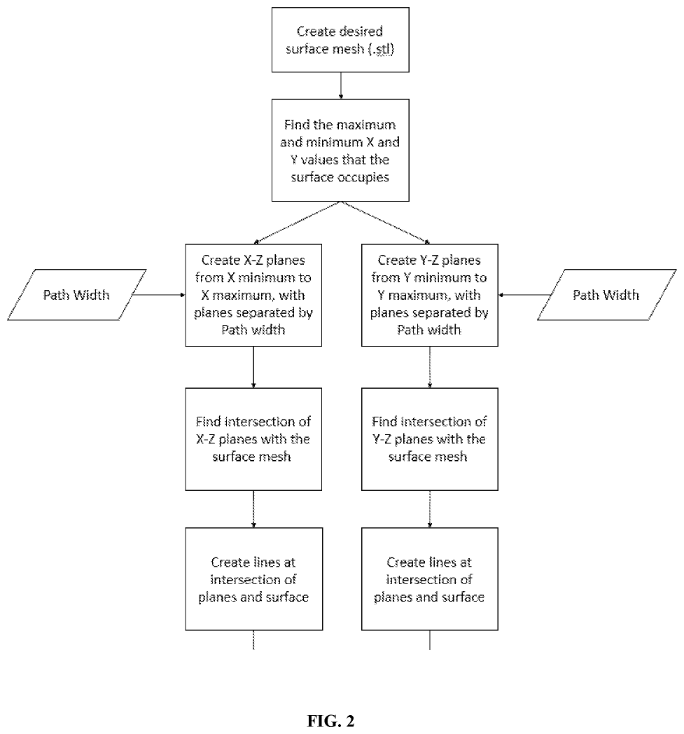

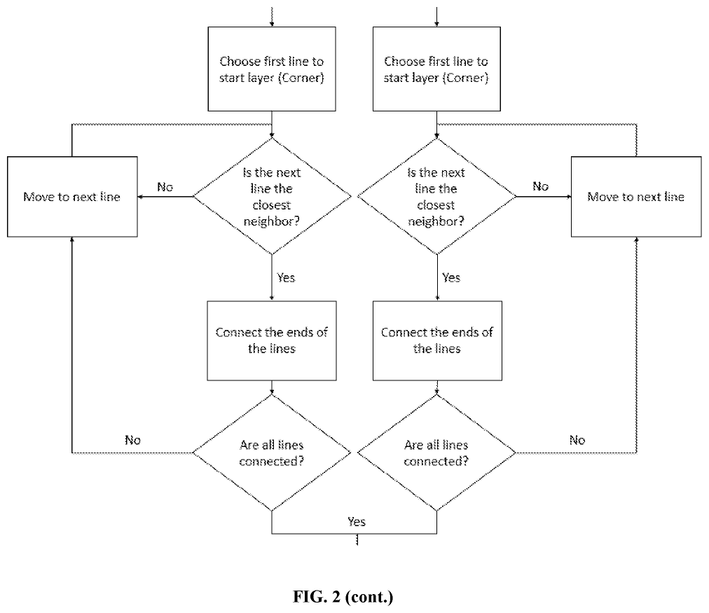

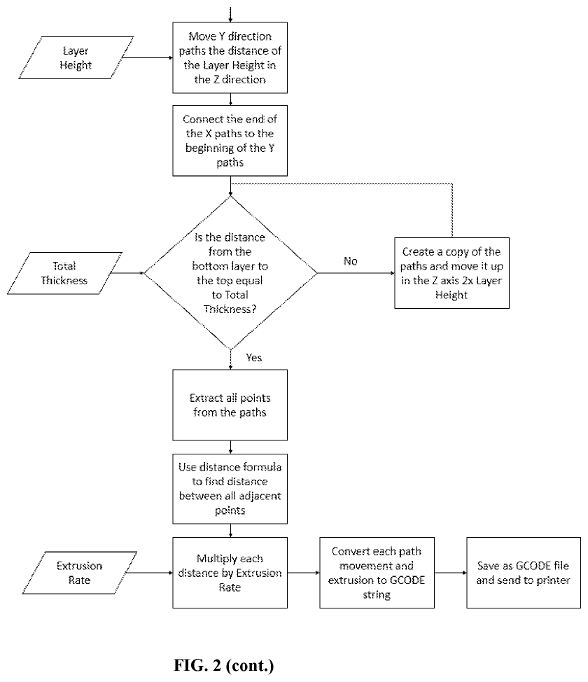

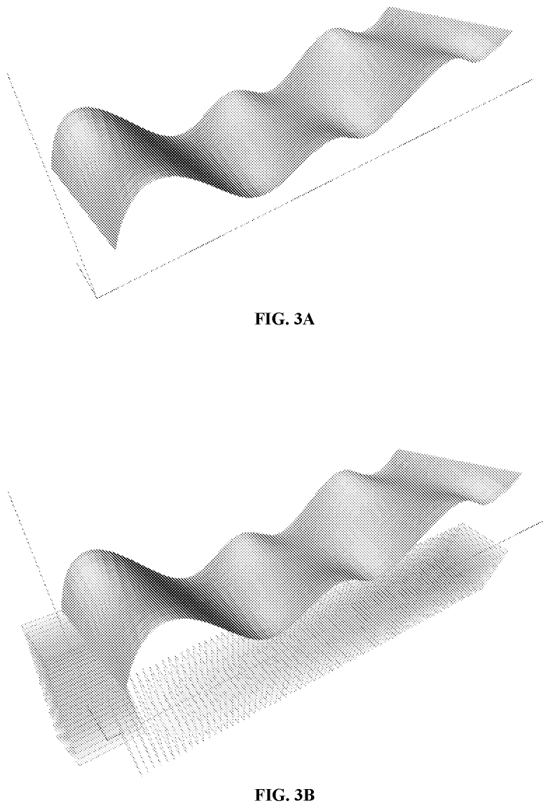

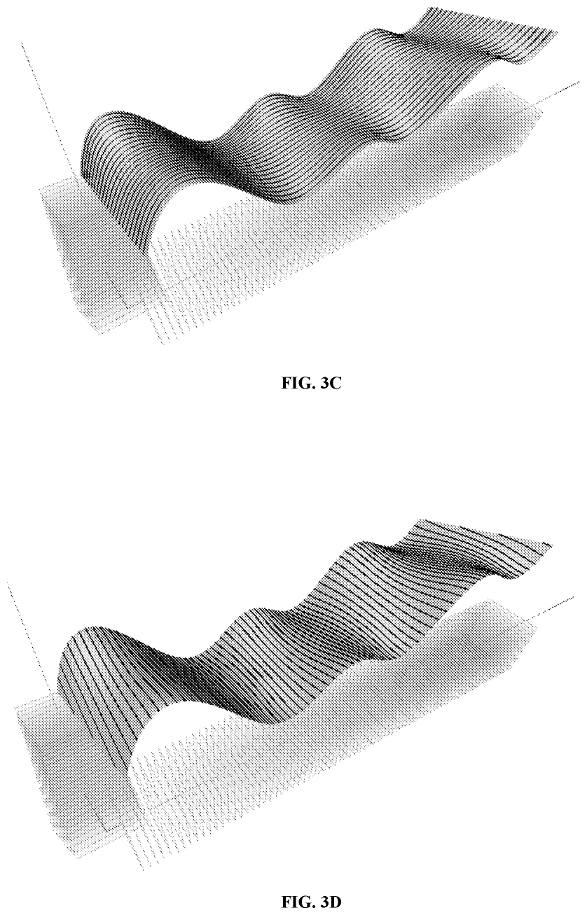

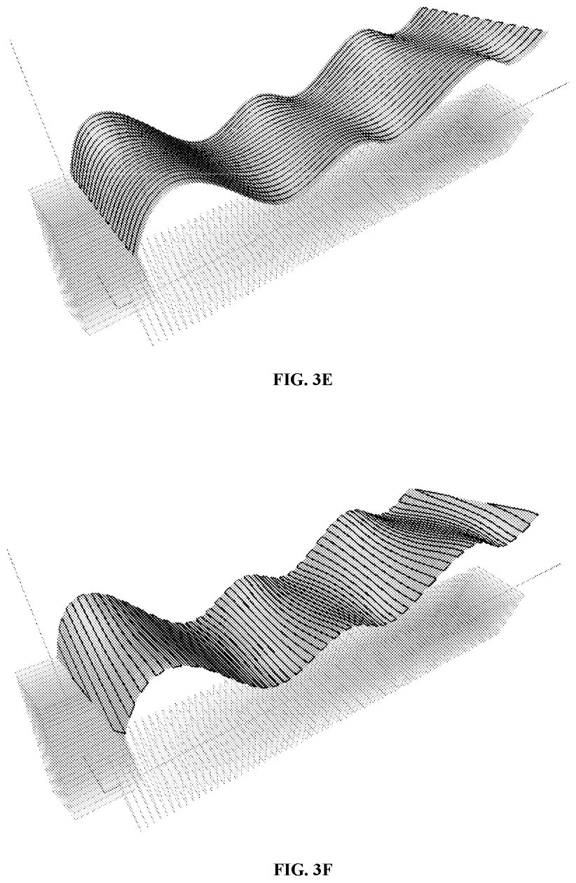

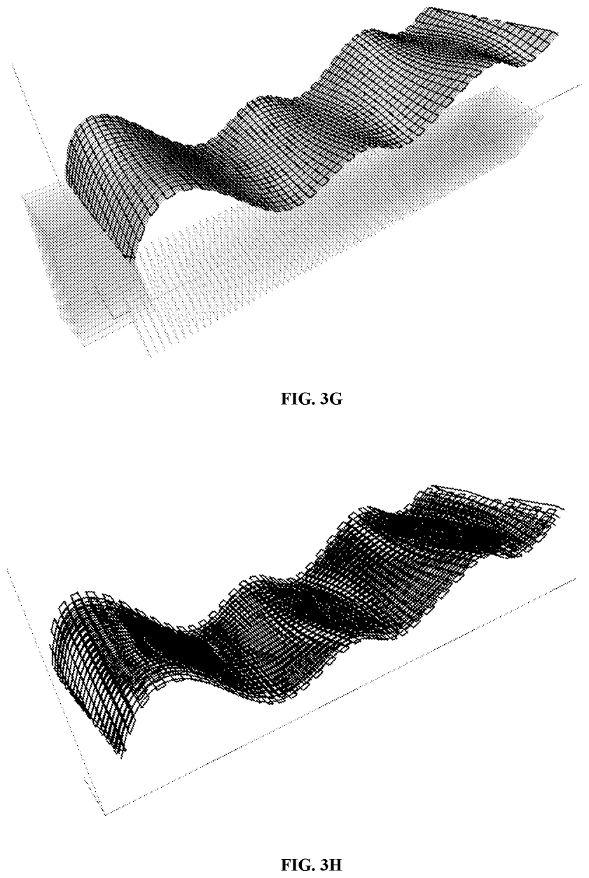

Creating X-Z and Y-Z planes to obtain lines of intersection between the surface and these planes from a non-planar surface model of an article to be fabricated to obtain X and Y coordinates of the surface, and correlating Z values of the surface to the X and Y coordinates to create non-planar print paths, is provided in further illustration in FIG. 2 and FIGS. 3A-H.

In some embodiments, X and Y coordinates of the surface of a non-planar model of an article to be fabricated are created to create an X-Y plane. Planes orthogonal to the X-Y plane are also created to obtain lines of intersection between the surface and the planes. In some embodiments, the X-Y plane comprises X-Z and Y-Z planes. Coordinates of intersecting lines are correlated to create non-planar print paths. In some embodiments, the non-planar print paths are offset to create alternating print paths to a desired thickness of the article. In some embodiments, printing distance of a print head to a print substrate is calculated based on the distance between sequential points on the print paths. In some embodiments, points that comprise the print paths are converted to strings, and the strings are combined with extrusion values to create lines of a GCODE file.

With reference to FIG. 2, illustrated is a flowchart of one embodiment of an algorithm used to define print paths for carrying methods of fabricating an article disclosed herein. The algorithm begins by creating a desired surface mesh of the surface of a non-planar surface model of an article to be fabricated. This is done by finding the maximum and minimum X and Y values that the surface occupies. The “Path Width,” meaning the width of the path to be printed by an extrusion printer, is then used to (i) create X-Z planes from X minimum to X maximum, with planes separated by path width and to (ii) create Y-Z planes from Y minimum to Y maximum, with planes separated by path width. (Note: Path width is a parameter that a user determines before setting up any print, so in the algorithm, a predetermined value for path width is used. Also, factors such as path width and layer height do not need to remain constant throughout a print.) Parallel instructions paths are taken for the X-Z planes and the Y-Z planes. This involves finding the intersections of X-Z planes with the surface mesh and finding the intersections of Y-Z planes with the surface mesh, and then creating lines at the intersection of the planes and surface. The first line must then be chosen to start a print layer, which is, generally, the corner. If the next line is the closest neighbor, then the ends of the lines are connected. If the next line is not the closest neighbor, then the algorithm checks the next line, and so on until the next line is the closest neighbor, at which point the ends of the lines are connected. If all the lines are not connected, then the algorithm is instructed to move to the next line, the closest neighbor, and connects the ends of the lines until all lines are connected.

Once all lines are connected for both the X-Z planes and Y-Z planes, layer height is directed by moving Y direction paths the distance of the layer height in the Z direction. The end of the X paths are then connected to the beginning of the Y paths. If the distance from the bottom layer to the top is equal to the total thickness, then all points are extracted from the paths. If, on the other hand, the distance from the bottom layer to the top are not equal to the total thickness, a copy of the paths is created and moved up in the Z axis 2× the layer height until the distance from the bottom layer to the top is equal to the total thickness.

A distance formula is then used to find distance between all adjacent points. An extrusion rate is then instructed by multiplying each distance by an extrusion rate, converting each path movement and extrusion to a GCode string, and then saving as a GCode file and sending to the printer.

Turning now to FIGS. 3A-H, shown is a series of computer-generated images showing steps involved in generating Gcode files for non-planar prints according to embodiments of a method of fabricating an article described herein. FIG. 3A shows a surface model of a three-dimensional article to be fabricated. FIG. 3B shows a silhouette of intersecting X-Z and Y-Z planes, created between minimum and maximum X and Y coordinates of the surface, spaced equal to the Path Width. FIGS. 3C-D show mapping of paths at plane-surface intersections. FIGS. 3E-F shows connection of nearest neighbor paths. FIG. 3G shows a combination of X paths and Y paths. FIG. 3H shows that alternating layers are repeated to achieve the desired thickness with layers spaced at increments equal to the Layer Height.

In carrying out the article fabrication methods of the present disclosure, printing may be carried out using an available 3D printing system, which are well-known in the art, and which may be modified to accommodate unique fabrication methods disclosed herein. For example, one suitable 3D printing system suitable for carrying out methods disclosed herein is the Ender 3 (Creality) printer. Modifications to the 3D printer may be made to accommodate the methods disclosed herein. As examples of such modifications, the stock motherboard may be replaced with, e.g., a MKS Gen L v1.0 for dual extruder capabilities. Also, a second Z-axis motor may be added to stabilize both ends of the gantry during vertical motion. Two Replistruder V3 (Lee et al., “3D Bioprinting of Collagen to Rebuild Components of the Human Heart,” Science 365(6452):482-487 (2019), which is hereby incorporated by reference in its entirety) extruder systems may be modified to attach to the printer gantry.

In some embodiments of the methods disclosed herein, printing of an article or object is carried out using methods of embedded printing. FRESH is an example of a freeform reversible embedding (FRE) technique for fabricating an object. FRE techniques are additive manufacturing (AM) processes by which a structure material is deposited and embedded into a support material (referred to as a “support bath,” in some instances) that physically supports and maintains the intended geometry of the embedded structure material during the manufacturing process. Although the techniques described herein are primarily discussed in terms of the FRESH process, this is merely for illustrative purposes and it should be understood that the techniques are generally applicable to any FRE process. In one implementation of a FRE process, the structure material can be deposited via an extruder assembly, which can include a syringe housing the structure material and a needle through which the structure material is extruded. In some embodiments, the extruder assembly can further including a gantry supporting the syringe, a motor assembly or other movement assembly configured to translate and/or rotate the gantry, the syringe, and/or the platform on which the support material rests, and an actuator (e.g., a motor) configured to depress a plunger to extrude the structure material from the syringe through the needle tip into the support material as the needle is translated through the support material to form a 3D object. After deposition of the structural material has been completed, the support material is then removed to release the 3D object. As with other AM techniques, the 3D object fabricated by the extruder assembly is based upon a computer model. The computer model is sliced into a series of layers (e.g., by Skeinforge or KISSlicer software), which are then utilized to generate a set of instructions (e.g., GCode instructions) for controlling the movement of the extruder assembly to form the 3D object defined by the computer model from the structure material.

Thus, other aspects of the present disclosure include a system and/or a computer program product that functions to control a system to carry out the methods of the present disclosure.

### Computer Program Products

The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

As discussed above, certain aspects of the present disclosure are described herein with reference to flowchart illustrations and/or block diagrams of methods, apparatus (systems), and computer program products. It will be understood that each block of a flowchart illustration and/or block diagram, and combinations of blocks in a flowchart illustration and/or block diagram, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowcharts and/or block diagram block or blocks.

The computer readable program instructions may also be loaded onto a computer, other programmable data processing apparatus, or other device to cause a series of operational steps to be performed on the computer, other programmable apparatus, or other device to produce a computer implemented process, such that the instructions which execute on the computer, other programmable apparatus, or other device implement the functions/acts specified in the flowchart and/or block diagram block or blocks.

The flowcharts and block diagrams disclosed herein illustrate the architecture, functionality, and operation of possible implementations of systems, methods, and computer program products according to various embodiments of the present disclosure. In this regard, each block in the flowcharts or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### Devices and Systems for 3D Printing

Methods of the present disclosure involve fabricating an article. In some embodiments, fabrication of an article is carried out by means of printing a material onto a substrate or into a support bath to form the article.

In some embodiments, fabricating an article according to methods disclosed herein is carried out using a 3D printer, which prints objects by depositing materials using a printer head. In some embodiments, the methods disclosed herein are carried out by when a 3D printer, e.g., comprising a material deposition tool is controlled by an external computer that controls dispersion of material from a material deposition tool and, optionally, relay information pertaining to sensors positioned on a material deposition tool.

In some embodiments, printing according to the method of the present disclosure is carried out by extrusion of a material from a print head. Any material capable of being extruded may be used in the methods of the present disclosure. Also, combinations of materials, either combined in a print head and extruded out of a single nozzle, or extruded separately from different nozzles of one or multiple print heads, may be used in printing systems for carrying out the methods of the present disclosure.

In some embodiments, the material extruded from a print head to fabricate an article is a solution.

In some embodiments, the print head is moveable in X, Y, and Z directions.

Fabrication of an article using a 3D printing system may be carried out as material is deposited from a material deposition tool (also referred to as an extrusion tool, a print head, and/or a nozzle) in a pattern on a substrate. In some embodiments, the pattern is established by a system controller according to methods and algorithms described herein, which operate according to an electronic data file so that changes to the article only require changes to the design data of the system controller. In its basic operation, a transfer device, according to a manufacturing plan programmed into the system controller, operates an extrusion tool to carry out the fabrication steps by depositing material in a designated pattern along a substrate or into a support bath.

In some embodiments, methods disclosed herein may be carried out in an enclosed environment, although this is not required. An enclosure may primarily serves two purposes: safety and manufacturing environment control. An enclosure may include fume extraction filtration and/or ducting connections to external ventilation in order to allow use of the system in a human occupied space. An enclosure may also be used to prevent human operators from contact with high temperatures, high voltages, laser radiation, etc.

An enclosure also permits control of the ambient environment in which fabrication takes place. Temperature, humidity, and gas mix of the environment, as well as illumination can all be controlled over time. This permits a fabrication system to control the evolution of the properties of deposited materials over time. For instance, the system can maintain a low temperature to limit chemical reaction until all of a certain material has been deposited, then cause reaction in the deposited material by elevating the temperature, or viability of living tissues being deposited can be enhanced by maintaining incubating conditions.

For certain purposes, especially when working with very sensitive materials, or under very stringent sterility conditions, it may be desirable to include a “local enclosure” around the object being fabricated. This can take the form of a sterile and/or sterilizable membrane or bag which encloses the active region of the substrate, and which can be penetrated by the tip of a tool in a selective fashion. This arrangement permits the upper surface of the membrane to move with the tool while the lower surface remains stationary relative to the build surface and the object being fabricated. Such local enclosures can, for instance, permit use of a version of the fabrication system not specifically designed for sterile work to work with materials which require high standards of sterility, further enhancing the breadth of utility of the methods disclosed herein.

In some embodiments, the system controller is operated by fabrication system software that automatically converts design data into a manufacturing plan. In some embodiments, the fabrication system software automates the majority of operations of the system, permitting the designer's intent to be converted into a realized product with a minimum of labor and prerequisite specialized knowledge about the materials being used (i.e., deposited). The system controller may be designed to store data about system performance and to improve the system automatically by updating data and models which it uses for manufacturing planning, manufacturing simulation, manufacturing plan execution, and operation control.

In some embodiments, the main components of the system software of the system controller are the manufacturing planning, operation control, materials/tools database, and design database. The manufacturing planning software is responsible for converting the designer's intent, via the product description data, into an executable plan for producing the design.

In the simplest approach, the sorting of paths will arrange the toolpaths for printing. More sophisticated optimizations take into account more detailed information about the materials, tools, performance of the system itself, and known design rules (e.g., from the design database) for certain aspects of the design. More sophisticated optimizations can also involve optimizing the shape and directionality of material deposition paths to achieve desired properties in the finished product(s). This can include optimizing mechanical properties such as tensile strength along primary stress axes, or possibly even along 3-dimensional curves through an object being fabricated. Additionally, some biomaterials need to be shape agnostic so long as they can deform within a prescribed operating domain. A simple example is the sail on a boat. While a single sheet of fabric, it can assume many “shapes” without tearing. Heart valves are a human example of this. It isn't necessarily that they must retain a single shape, but be manufactured so that they can originate and operate in a complex shape continuum. There are “hard” tissue examples of curved tissue shapes that would be made more accurate/biomimetic with curvilinear printing approaches, like the head of a bone or the labrum, but those cases would not need to deform as much. The method of the present disclosure is capable of achieving all these options, as being able to print curvilinear fibrous tissues improves both types applications.

These microstructures can also be determined and replicated through direct inspection of the native biological (or non-biological) material microstructure themselves, for example though CT, MRI, or Optical imaging. One could also design custom material compositions/orientations, for example as a patch to adjust local mechanical strain fields that a tissue is exposed to. These “axes” need not be articulated as orthogonal or Euclidean, and the algorithm described herein is not constrained in this way despite operating in an “XYZ” framework as many different material configurations can be transformed into Cartesian coordinates and vice versa. Reference herein to X-Y coordinates or X-Y planes should not be interpreted as only meaning a flat plane of Cartesian coordinates. Methods of the present disclosure are not constrained to formal X-Y flat planes, but may also include X-Y curved planes or surfaces. While it may be somewhat typical for an “X-Y plane” to mean a flat surface, the axes of X and Y could be rotated away from horizontal. Coordinate transformations to Cartesian is currently ideal for the Cartesian printer setup, but it is immaterial, for example, if the printer is designed to have a polar printing axis or other conventions. The principles are the same. The methods disclosed herein are adaptable to almost any coordinate transformation or curved surface that does not double back or fold in on itself (for example making a knot). Thus, the term “X-Y plane” does not simply mean a flat surface, which may the prevailing bias of current print technologies owing to horizontal print surfaces that are used and gravity.

This type of final material property optimization can also be used to provide preferential axes for tissue growth when working with living biological materials. Information to drive these optimizations can be extracted from the materials/tools and design databases, from realtime and historical performance data that the system maintains about itself, and from a simulation of the manufacturing process. This latter method becomes more crucial as designs, material behavior, and material interactions become more complex, and specifications for the finished product become more stringent. The simulation may make use of models of material evolution over time and in response to environmental conditions which are stored in the materials/tools database, models of system operation and performance, and may perform physical simulations of a candidate manufacturing process for a given design (including finite element models, or other physics-based modeling). In this way, the system can make predictions about the quality and/or performance of a final product that might result from a given manufacturing plan, which can then be used for automated searching for manufacturing plans which satisfy the objective functions supplied by a user in the design specifications for the object to be produced. The simulation, in an interactive mode, can permit a designer to explore the effect of alternative designs on the complexity of the manufacturing process. The simulation of the manufacturing process also plays an essential role for error recovery/correction during a manufacturing process. Error recovery/correction is important because producing complex objects with novel or experimental materials is costly and time consuming. Further, if the system does not detect and recover from errors during the manufacturing process, the errors will—by the very nature of the process—may be buried within the object being produced and be difficult or impossible to diagnose and repair. The manufacturing simulation allows the system, at any step in the manufacturing process, to compare the state (geometry and other automatically detectable properties) of the simulation to the state of the real object being produced as measured by 3D scanning (or other) sensors. Discrepancies can be remedied by generating modifications to the manufacturing plan which replace or circumvent the error—these too can be explored in simulation, and the effect of the errors and the modifications to the manufacturing plans can be used to update predictions of the quality and performance of the final product.

The manufacturing planning software can include special purpose data conversion capabilities to assist the designer in conveying design data to the system. This can include automatic conversion of computed tomography and magnetic resonance imaging data into manufacturing plan geometry data, or automatic conversion of point cloud data resulting from non-contact scanning directly into manufacturing plan geometry data and local material properties, for example as revealed by different contrast intensities and/or patterning inside the geometry.

In some embodiments, methods disclosed herein are carried out using a 3D printer, which is a standard existing printer (for example, designed to print parallel slices to create the 3D object, one slice at a time, from lower layers to higher layers), but which is programmed with improved printing paths based on the improvements described herein.

As discussed above, a computing unit may be integrated with the 3D printer, for example, as a control console and/or control unit and/or instructions code stored within the 3D printer. Alternatively or additionally, the computer unit may be implemented as a unit external to the 3D printer, that creates the print paths generated by an algorithm as described herein that are provided (e.g., transmitted using a communication channel such as a network, or obtained from a storage device) to the 3D printer for use in guiding the printing of the 3D object. For example, the computing unit may be implemented as, for example, a client terminal, a server, a mobile device, a desktop computer, a thin client, a Smartphone, a Tablet computer, a laptop computer, a wearable computer, glasses computer, or a watch computer. The computing unit may include locally stored software that performs one or more of the acts necessary to the printing process and/or may act as one or more servers (e.g., network server, web server, a computing cloud) that provides services (e.g., one or more of the acts necessary to the printing process) to one or more client terminals, for example, providing software as a service (SaaS) to the client terminal(s), providing an application for local download to the client terminal(s), and/or providing functions using a remote access session to the client terminals, such as through a web browser.

The processing unit may be implemented, for example, as a central processing unit(s) (CPU), a graphics processing unit(s) (GPU), field programmable gate array(s) (FPGA), digital signal processor(s) (DSP), or application specific integrated circuit(s) (ASIC). The processing unit(s) may include one or more processors (homogenous or heterogeneous), which may be arranged for parallel processing, as clusters, and/or as one or more multi core processing units.

A program store may be used to store code instructions implementable by the processing unit, for example, a random access memory (RAM), read-only memory (ROM), and/or a storage device, for example, non-volatile memory, magnetic media, semiconductor memory devices, hard drive, removable storage, or optical media (e.g., DVD, CD-ROM). For example, the program store may store code instructions that executes one or more acts of the fabricating methods described herein.

The computing unit may include a data repository for storing data, for example, a computer aided design (CAD) application for use by a user to design the geometric object for printing. The data repository may be implemented as, for example, a memory, a local hard-drive, a removable storage unit, an optical disk, a storage device, and/or as a remote server and/or computing cloud (e.g., accessed using a network connection). It is noted that code instructions may be stored in the data repository, for example, with executing portions loaded into the program store for execution by the processing unit.

The computing unit may include a network interface for connecting to a network, for example, one or more of a network interface card, a wireless interface to connect to a wireless network, a physical interface for connecting to a cable for network connectivity, a virtual interface implemented in software, network communication software providing higher layers of network connectivity, and/or other implementations. The computing unit may access one or more remote servers using a network, for example, to download the initial definition of the geometric object, and/or to provide a generated set of printing instructions.

The computing unit may connect using a network (or another communication channel, such as through a direct link (e.g., cable, wireless) and/or indirect link (e.g., via an intermediary computing unit such as a server, and/or via a storage device) with one or more of:

A client terminal(s), for example, when the computing unit acts as a server providing SaaS.

A remotely located additive manufacturing system that manufactures the geometric object based on the algorithm described herein. It is noted that a 3D printer may be locally located and connected to the computing unit (e.g., integrated within, connected by a cable, connected by a short range wireless communication protocol, connected by a local network), and an AM system may be remotely located and connected to the computing unit using a network.

A storage unit that stores the initial definition of the geometric object (e.g., provided by a client terminal, server, and/or used by the computing unit for remote storage), and/or the generated set of univariate curves (e.g., created by the computing unit for use, for example, by the manufacturing system for manufacturing the 3D object). The storage unit may include, for example, a storage server, a computing cloud storage server, or other implementations.

In some embodiments, the computing unit includes or is in communication with a user interface allowing a user to enter data and/or view presented data. Exemplary user interfaces include, for example, one or more of, a touchscreen, a display, a keyboard, a mouse, and voice activated software using speakers and microphone.

One or more acts of the method described herein may be implemented by code instructions stored in the program store (and/or stored in a data repository) executed by the processing unit of the computing unit.

Another element of a system controller is operation control, which includes all of the feedback control, self-testing, data-collection software required to operate the system hardware and execute a manufacturing plan produced by the manufacturing planning software, including: (i) low-level control software for tools and material cartridges which may be executed by computational hardware within the tools and cartridges themselves; (ii) the feedback control laws which operate the environmental controls within the system enclosure; (iii) the motion control software that commands the positioning systems and coordinates positioning system control with tool deposition control; (iv) control and path planning for the transfer device used for tool and material changes; and (v) scanning sensor control, data collection, and data conversion.

The system controller may be used to store information about the properties of materials used in fabrication, the types and shapes of deposits of material that can be made by a given deposition tool, specialized parameters for controlling deposition tools to achieve each type of deposit, material interactions with other materials, and other information. The material properties information may be in the form of a complex material model, which can include time evolution of the material properties in response to conditions or material property changes with respect to position and orientation. This type of information may be used in many ways in manufacturing planning and manufacturing simulation, including: (i) generating plans for the control of material cartridges and enclosure environmental conditions to guide the evolution of material properties before, during, and after deposition (e.g., cellular reproduction rates, chemical reaction rates, etc.); (ii) identifying complications in a manufacturing plan, e.g., where adjacent materials might be reactive, where a liquid might need to be deposited only after a solid boundary has been constructed to contain it; and (iii) identifying how long manufacturing plan steps must be spaced by in order to allow deposited materials to solidify, react, etc.

The information in the database is compiled from a variety of sources. First, the embedded intelligence, sensing, and communications in material deposition tools and cartridges obtains realtime information on quantity and status of materials loaded into the system for use in the current manufacturing plan, and health and operational status of the tools. Second, chemical or materials data references and research obtain general chemical and materials properties data and dynamic properties models. Third, manual calibration by operators or machine learning algorithms within the system (See Malone et al., “Application of Machine Learning Methods to the Open-Loop Control of a Freeform Fabrication System,” Proceedings of the 15th Solid Freeform Fabrication Symposium, Austin TX, August 2004, pp. 377-388, which is hereby incorporated by reference in its entirety) retrieve tool control parameters required to generate a given material deposit geometry.

The design database is a repository of subunits of manufacturing plans and design rules of thumb that have been demonstrated to be successful, as well as complete designs and manufacturing plans for useful functional modules that are likely to be desirable inclusions into other designs. Maintaining this database is important, because no practical manufacturing simulation will be able to predict all issues of concern during manufacturing. The successful designs, rules, tricks, and modules capture much of the hidden information that would be difficult or impossible to simulate.

Modules can be complete functional devices, such as batteries, actuators, joints, transistors, etc., which can be freeform fabricated directly into other designs to produce a composition of higher functionality. Design rules can be empirical relations between, for instance, the volume of active material deposited in a certain type of device, and the performance of the resulting device.

In order for the manufacturing planning and control system to be able to automatically monitor the progress of a fabrication operation, to identify and locate objects placed in the build environment, and to measure the geometry of the finished product, the system may include a non-contact ranging sensor device which can be scanned by the positioning system across the build surface (i.e., substrate). In some embodiments, this device has a distance resolution of 10 micrometers, while the positioning system has an XY-plane resolution of 5 micrometers, although other distance resolutions and XY-plane resolutions may also be suitable. The benefit of this type of sensor is that it is compact enough to embed within material deposition tools near the point of material deposition (e.g., nozzle), and of low enough cost to use in each cartridge, even in multiples. The presence of the ranging sensor within a material deposition tool permits the acquisition of the geometry of deposited material with minimal interruption of the fabrication process (e.g., without pausing manufacturing in order to mount a separate sensing tool to perform the scanning) and very near the point of deposition. This improves the value of the resulting data for use in feedback control of the deposition tool-geometric flaws in deposited material can be detected quickly, minimally allowing the manufacturing planning software to design compensating material deposits which can be executed in a timely fashion, without compromising the properties of materials which are sensitive to the time since deposition. If such sensing can be provided in a 360 degree circle around and very proximate to the point of deposition, such sensing could be used for online feedback control of the deposition tool-allowing the control law for the tool to adjust tool actuator commands in order to achieve the desired deposit geometry. Materials may be assessed rheologically by known methods at a point or small local volume, even if not fully solid. It may or may not be necessary to measure such factors in real time, but they could be measured in real time if necessary. Hybrid contact methods like scanning ion conductance can return mechanical as well as positional values, but across a viscous material surface. As would be appreciated by a person of ordinary skill in the art, scanning ion conductance can be employed in a couple of different modes to detect surface mechanics. Even if not useful for online feedback control, more frequent “semi-online” feedback may greatly improve the achievable final quality of the object being fabricated, because it allows the manufacturing planning software to compensate for variability in the properties of the materials being employed, or other deposition errors by automatically generating modified manufacturing plans which compensate for the errors.

### Fabrication Materials

Suitable deposition materials for carrying out methods of the present disclosure include, without limitation, any material capable of being deposited from a material deposition tool onto a substrate. The type of material used in any of the methods disclosed herein will depend on the type of material deposition tool being employed and the article to be fabricated. For example, when a fusible-material extrusion tool is employed, suitable materials include, without limitation, thermally liquefied plastics, low melting-point metals, and other fusible materials. When a syringe cartridge is employed, suitable materials include, without limitation, virtually any liquid, slurry, or gel.

Using a given material with a solid freeform fabrication system typically requires modifying that material somewhat to make it useable by a tool in a given fabrication system, or at an extreme, development of an entirely new tool. If it is desired that the new material should be able to be incorporated into designs made with a variety of materials, then a significant amount of effort is required to ensure compatibility of materials and processes, to develop operating parameters for tools, and to fully characterize the resulting materials, deposits, and generate all of the data required to enable the system to make full use of the material. For this reason, each successful material formulation is a significant achievement in itself.

Methods such as molecular self-assembly may be important factors in carrying out methods of the present disclosure, permitting localized self-assembly of molecular level structures. Ink-like materials can be made that are MEMS devices dispersed in a liquid carrier. The MEMS devices can be deposited as a normal ink. After evaporation of the carrier, the devices can be electrically connected by depositing electrically conducting materials in appropriate patterns.

In some embodiments, co-extrusion nozzles are used where, for example, material A and material B are deposited simultaneously, either as a mixture or one enveloping the other. In some embodiments, such nozzles may be spinning nozzles, which produce a helical deposition shape. The methods of the present disclosure are able to accommodate whatever nozzle/crosslinking approach known or that may be developed, as the algorithm of the present disclosure is directed to specifying the path a print head travels, although other features discussed herein could be controlled according to or along path profiles to improve outcome.

Novel applications of the methods disclosed herein in the areas of biomedical implants can include controlling the biocompatibility of conventionally manufactured biomedical implants by depositing compatible living tissues, or appropriate chemicals, as a covering for the devices. Novel biomedical implants can be produced which are hybrids of freeform fabricated materials—both biological and non-biological—with conventionally manufactured devices which are embedded within. This can result in implants which have functionality that is unachievable by conventional manufacturing means.

Implanted materials may be completely non-biological. Such implants may be improved by the methods disclosed herein. The same is true for non-implanted materials.

In some embodiments, material that may be used in fabricating articles according to methods disclosed herein is a material having seeded cells, such as a hydrogel having seeded cells. Suitable hydrogels may include, without limitation, alginate, agarose, collagen, chitosan, fibrin, hyaluronic acid, carageenan, polyethylene oxide, polypropylene oxide, polyethylene oxide-co-polypropylene oxide, hydroxypropyl methyl cellulose, poly(propylene fumarate-co-ethylene glycol), poly(ethylene glycol)-co-poly(lactic acid), poly(vinyl alcohol), KDL12 oligopeptides, and poly(n-isopropyl acrylamide).

In some embodiments, the hydrogels have a controlled rate of crosslinking through the adjustment of environmental variables including, but not limited to, temperature, pH, ionic strength, heat, light, or the addition of chemical crosslinking agents such as calcium, magnesium, barium, chondroitin, sulfate, and thrombin. In some embodiments, the cross-linking compound is provided in a weight ratio of hydrogel to cross-linking compound of about 1:100 to 100:1, respectively. All of these elements could be controlled along the more complex curvilinear print path of the methods disclosed herein, which constitute an important new director of fabrication.

In some embodiments, cells in the hydrogel are of a single cell type. Suitable cell types include, without limitation, all mammalian or plant cells. In some embodiments, the cells include one or more type of cell selected from chondrocytes, osteoblasts, osteoclasts, osteocytes, fibroblasts, hepatocytes, skeletal myoblasts, cardiac myocytes, epithelial cells, endothelial cells, keratinocytes, neurons, Schwann cells, oligodendrocytes, astrocytes, pneomocytes, adipocytes, smooth muscle cells, T cells, B cells, marrow-derived stem cells, hematopeotic stem cells, osteoprogenitor cells, neural stem cells, and embryonic stem cells.

In some embodiments, cells in the hydrogel may be of more than one cell type.

The methods of the present disclosure are relevant to the fabrication of heterogenous materials, such as, for example, where there is more of material A than material B in some areas, or where fibers of material A are present in bulk of material B, combinations, as well as more/different cells in different areas. Cellular domains may also be specified with fibrous non-cellular components. Such factors only depend on the number of nozzles for material deposition available to a given fabrication system. In some embodiments, the methods of the present disclosure encompass the creation of fibrous bulk materials by extruding through multiple nozzles. In some embodiments, the methods of the present disclosure encompass printing curvilinear fibers inside a curved bulk where, for example, three (or more) different materials undulate on different axes.

In some embodiments, dispensing material from the material deposition tools is carried out under sterile conditions. In some embodiments, dispensing of material may be carried out in a hermetically sealed envelope.

The methods disclosed herein may further involve determining geometry and cell distribution of the article prior to carrying out the printing or material dispensing step. A system controller may also be programmed with instructions effective to cause the transferring and dispensing steps to be carried out to produce an article with a desired geometry and cell distribution.

In some embodiments, the determined geometry is free-form.

In some embodiments, the geometry is an anatomic shape, even patient-specific.

To determine geometry, a computerized scan of a tissue/organ may be generated, such as a scan of a tissue/organ to be replaced. The geometry to be fabricated can be determined from any method capable of generating 3D data sets, including, without limitation, 3D laser scanning, confocal microscopy, multi-photon microscopy, computerized tomography, magnetic resonance imaging, ultrasound, and angiography.

In some embodiments, after a hydrogel with seeded cells is fabricated pursuant to the methods disclosed herein, the article may be incubated under conditions effective to grow the cells. In some embodiments, incubation may be carried out on the substrate, or the article may be transferred to a new substrate for more optimal growth conditions.

Some aspects of the present disclosure relate to a method of fabricating a living tissue. This method involves providing a plurality of liquid compositions including a composition having a hydrogel with seeded cells. The plurality of compositions are selected in a selected sequence suitable to form the living tissue. The compositions are dispensed in the selected sequence and in a pattern suitable to fabricate the living tissue according to instructions from an algorithm disclosed herein.

Suitable liquid compositions for carrying out such methods include hydrogels, with or without seeded cells, which in some embodiments, contain cross-linking compounds to provide structure to the fabricated tissue.

As noted above, in some embodiments, methods disclosed herein are carried out using the FRESH process. In some embodiments in carrying out the methods using embedded printing, the structure material includes hydrogels. The hydrogels can be formed from extracellular matrix (ECM) materials, such as natural polymers (e.g., collagen), polysaccharides (e.g., alginate or hyaluronic acid), glycoproteins (e.g., fibrinogen), decellularized ECM materials, and ECM-based materials (e.g., Matrigel, which is a mixture of structural proteins such as laminin, nidogen, collagen, and heparan sulfate proteoglycans, secreted by Engelbreth-Holm-Swarm mouse sarcoma cells). In some embodiments, the hydrogel structure material can be formed from decellularized ECM materials or tissue harvested from the patient's biological structure being replaced or augmented with the FRE-fabricated object. In this way, the properties of the object can be precisely tailored to the properties of the biological structure at issue. In some embodiments, the support material can include a Bingham plastic or Bingham plastic-like material. Such materials behave as a rigid body at low shear stresses but flow as a viscous fluid at higher shear stresses. Accordingly, the support material provides little mechanical resistance to the needle as it is translated therethrough, but physically supports and holds in place the deposited structure material. Thus, the support material can maintain soft materials (e.g., the structure material) that would collapse if they were printed outside of the support material in the intended 3D geometry. As one example, the support material can include a slurry of gelatin microparticles processed to have a Bingham plastic rheology, as described in Hinton et al., “Three-dimensional Printing of Complex Biological Structures by Freeform Reversible Embedding of Suspended Hydrogels,” Science Advances 1: e1500758 (2015), which is hereby incorporated by reference in its entirety). In some embodiments, the support material can be tailored to match the gelation mechanism of the structure material, such as exposure to divalent cations (e.g., Ca²⁺) for alginate or pH neutralization for collagen. In some embodiments, the support material can comprise a thermoreversible material. Accordingly, the 3D object can be released from the support material by heating the support material from an operational temperature (e.g., 22° C.) at which the 3D object is fabricated to a threshold temperature (e.g., 37° C.) that causes the support material to melt away from the object nondestructively.

In some embodiments, the object can be treated through various cross-linking techniques to selectively increase the rigidity of the overall object or portions thereof. In some embodiments, the step of inducing cross-linking in the structure material of the object can be skipped. In some embodiments, the support material can include a cross-linking agent for treating the structure material as it is deposited into the support material. For example, the support material can include divalent cations (e.g., 0.16% CaCl₂)) to induce cross-linking in the structure material while it is embedded in the support material. In another aspect, the structure material can be treated via a variety of different cross-linking techniques after the object has been released from the support material. For example, the released object can be treated with a cross-linking agent or via photo-induced cross-linking techniques (e.g., Photo-Induced Cross-Linking of Unmodified Proteins) to induce cross-linking of the support material.

Further, the amount or type of cross-linking can be selected based upon the type of structure material utilized to fabricate the object. For example, collagen may have a lower mechanical strength than alginate. To increase collagen's mechanical strength to match that of alginate, collagen structure material can be, for example, fixed for seven days in various concentrations of glutaraldehyde at 0.05% (v/v) and 0.5% (v/v) along with 1× phosphate buffered saline (PBS) to serve as the control along with standard alginate-fabricated objects fixed in 1% (w/v) CaCl₂). During testing, the mechanical properties of objects fabricated from different structure materials and with different amounts of cross-linking may be validated utilizing compression cylinders fabricated from the structure materials.

Thus, the FRESH process (and other FRE processes) generally include the steps of: (i) depositing a structure material into a support material according to a computer model of the structure to be fabricated, where the support material is configured to physically support and maintain the structure material in the intended 3D shape; (ii) removing the support material; and optionally (iii) cross-linking the structure material of the fabricated object either prior to or after the support material has been removed. Additional details regarding the FRESH process can be found in U.S. Pat. No. 10,150,258, which is hereby incorporated by reference in its entirety.

Embedded printing methods described herein are not limited to a hydrogel bath as the supporting material. Any material with effective viscosity/surface tension to maintain a stable support for extruded material could be used as a support material. Such materials would include, without limitation, an inorganic solvent, plasma, etc.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant additive manufacturing system will be developed and the scope of the term additive manufacturing system is intended to include all such new technologies a priori.

As used herein the term “about” refers to +/−10%.

The terms “comprises,” “comprising,” “includes,” “including,” “having,” and their conjugates mean “including but not limited to.” These terms encompasses the terms “consisting of” and “consisting essentially of.” The phrase “consisting essentially of” means that the method may include additional steps, but only if the additional steps do not materially alter the basic and novel characteristics of the claimed method.

As used herein, the singular form “a,” “an,” and “the” include plural references unless the context clearly dictates otherwise.

The word “exemplary” is used herein to mean “serving as an example, instance, or illustration,” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the present disclosure may include a plurality of “optional” features unless such features conflict.

Throughout this application, various embodiments of the disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicated number and a second indicated number and “ranging/ranges from” a first indicated number “to” a second indicated number are used herein interchangeably and are meant to include the first and second indicated numbers and all the fractional and integral numerals therebetween.

It is appreciated that certain features of the disclosure, which are, for clarity, described in the context of separate embodiments, may also be provided in combination in a single embodiment. Conversely, various features of the disclosure, which are, for brevity, described in the context of a single embodiment or some embodiments, may also be provided separately or in any suitable subcombination or as suitable in any other described embodiment of the invention. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant that all publications, patents, and patent applications referred to in this specification are to be incorporated in their entirety by reference into the specification, as if each individual publication, patent, or patent application was specifically and individually noted when referenced that it is to be incorporated herein by reference. In addition, citation or identification of any reference in this application shall not be construed as an admission that such reference is available as prior art to the present application. To the extent that section headings are used, they should not be construed as necessarily limiting. In addition, any priority document(s) of this application is/are hereby incorporated herein by reference in its/their entirety.

### EXAMPLES

The examples below are intended to exemplify the practice of embodiments of the disclosure but are by no means intended to limit the scope thereof.

### Example 1—Non-Planar Embedded Bioprinting for Complex Hydrogel Manufacturing Materials and Methods

### Non-Planar Print Slicer

The non-planar slicer was developed in the visual programming language in Dynamo Studio (Autodesk, Inc.). A surface of the desired geometry is imported into the script. Intersecting planes are generated in the X-Z and Y-Z axes at increments equal to the desired print path width. The planes are intersected with the surface to generate lines on the surface that represent the major paths. The ends of the major paths are connected by a nearest neighbor algorithm to generate an intuitive printing sequence of the paths. The paths generated by the X-Z and Y-Z planes are offset to create the first two layers of the print. These layers are duplicated in an alternating fashion to create the full, desired thickness of the print. Extrusion distance is calculated by the distance between sequential points on the paths. The points that comprise the path are converted to strings and combined with the extrusion values to create the lines of a GCODE file. Extruder changes are inserted into code where a material change is desired, and the resulting set of strings is exported to a GCODE file. Prints were sliced with 0.4 mm path width, 0.3 mm layer height, and six layers unless otherwise noted.

### Bioprinter

Printing was done using a modified Ender 3 (Creality) printer. The stock motherboard was swapped to a MKS Gen L v1.0 for dual extruder capabilities. A second Z-axis motor was added to stabilize both ends of the gantry during vertical motion. Two Replistruder V3 (Lee et al., “3D Bioprinting of Collagen to Rebuild Components of the Human Heart,” Science 365(6452):482-487 (2019), which is hereby incorporated by reference in its entirety) extruder systems were modified to attach to the printer gantry.

Printing with FRESH v2.0

Alginate (FMC Biopolymer) was prepared by dissolving 4% w/v in deionized water. 1% India ink was added for visualization in certain applications.

FRESH support bath was prepared as previously described (Lee et al., “3D Bioprinting of Collagen to Rebuild Components of the Human Heart,” Science 365(6452):482-487 (2019); Mirdamadi et al., “FRESH 3D Bioprinting a Full-Size Model of the Human Heart,” ACS Biomaterials Science & Engineering 6(11):6453-6459 (2020), which are hereby incorporated by reference in their entirety). Briefly, 20 g of gelatin (Fisher) was dissolved in 500 mL of distilled water and 500 mL of 100% ethanol at 40° C. 2.5 g of Pluronic F127 (Sigma) and 1.0 g of gum Arabic (Ward's Science) were subsequently dissolved in the solution. The pH was adjusted to 6 and stirred overnight while cooling to room temperature. The solution was aliquoted to 50 mL tubes and centrifuged at 400 rpm for 5 minutes. Supernatant was poured off and the remaining solution was resuspended in distilled water and centrifuged for 5 minutes at 1500 rpm.

Supernatant was again poured off and solution was washed with 0.1% calcium chloride or DMEM/F12 (Gibco) for alginate and collagen printing, respectively. A third centrifugation was done at 1500 rpm for 5 minutes and resuspended in either 0.1% calcium chloride or DMEM/F12. Solution was stored at 4° C. until printing.

Before printing, FRESH bath was mixed and placed in a vacuum chamber to come to room temperature. FRESH bath was then centrifuged at 1600 rpm for 5 minutes, the supernatant was poured off, and the FRESH bath was poured into the print container.

Post-print processing was performed by placing the print container into a 37° C. oven for at least an hour to melt the FRESH bath. Prints were then washed with 2% CaCl₂) at least three times to fully crosslink the alginate and remove excess FRESH.

### Multi-material Planar and BENT Printing

Two syringes of 4% alginate were prepared: one pure alginate and one with 1% India ink. An “M”-shaped surface was created for BENT slicing and an equivalent solid was created for slicing using Ultimaker Cura (Ultimaker BV). Prints were created using both slicers with three layers of black, three layers of clear, and three layers of black. Both were printed into a glass-walled container and a video of both prints was recorded. The print bath was then melted and washed away. Samples were then photographed.

### Print Angle Quantification

Prints were conducted by extruding 4% alginate into FRESH bath with a 25-gauge needle (McMaster-Carr) in a glass-paned vessel. Nine different angle prints were designed such that multiple lines (0°, 22.5°, 45°, 67.5°, 90°, −22.5°, −45°, −67.5°, −80°) could be produced in one print. In a cycle, the extruder translates horizontally, then upwards at the programmed angle, then horizontally, then down at the programmed angle, until the length of the vessel is spanned. Afterwards, the FRESH bath was melted in a 37° C. oven and washed with 2% CaCl₂). Images of the prints were taken through a microscope and analyzed in ImageJ.

### Printing and Mechanical Testing of Corrugations

Flat and corrugated prints were created that were 44 mm long and 16 mm wide. Corrugations were printed with six layers of 4% alginate with print paths running along the length of the geometry unless otherwise specified.

Samples were clamped onto a custom tensile test device and stretched to 20% strain at a rate of 1.0 mm/s. A force sensor measured the response to stretch, and the resulting stress was calculated based on the cross section of the samples. Initial modulus was calculated up to 10% strain for each sample and maximum modulus was found at the maximum slope of the stress-strain curve. Ultimate tensile strength (UTS) was measured by finding the stress at sample failure.

### Features of Corrugated Structures

Prints of varying amplitude, fillet radius, and size of corrugated features were created. Amplitude was varied by starting with a base model of five periods with 45 degree corrugations and reducing the amplitude of features by 0.5×, 0.25×, and Ox.

Fillet diameter was varied by taking the same base model and changing the baseline fillet diameter (0.5 mm) to 0 mm, 1 mm, and 2 mm. This effectively changes the sharpness of the print motion at the peaks and valleys of the geometry.

Size and frequency of corrugations was varied by changing the number of periods within the same length of geometry. The angle of corrugation was maintained at 45 degrees and fillet diameter was 0.5 mm. These geometries contain 0 (flat), 2.5, 5, and 10 periods of corrugation within them.

### Print Path Direction of Corrugated Structures

A five period corrugation with 0.5 mm fillets was sliced with the six print layers aligned in varying directions. Layers align parallel or perpendicular to the length (direction of mechanical stretch). Five prints were generated with 0, 2, 3, 4, or 6 layers aligned in the direction of stretch. Alternated layers were aligned perpendicular to the direction of stretch.

### Miura-Ori Origami Geometry

Miura-Ori (Silverberg et al., “Using Origami Design Principles to Fold Reprogrammable Mechanical Metamaterials,” Science 345(6197):647-650 (2014), which is hereby incorporated by reference in its entirety) surface geometries were created with the same dimensions as the simple corrugations. Geometries contained folds with depth of 0, 2, 4, and 6 mm. After printing and washing, samples were stretched to 20% strain for ten cycles. Initial modulus was calculated up to 10% strain for each sample and modulus was found at the maximum slope of the stress-strain curve. The Poisson ratio was calculated using the initial width and the width at 20% strain.

### Statistical Analysis

Data was analyzed using one way ANOVA followed by Tukey's multiple comparisons test. Values are presented as mean±standard deviation. Statistical significance is defined as P≤0.05 (*), P≤0.01 (**), P≤0.001 (***), or P≤0.0001 (****).

### Results and Discussion

### Development of BENT Slicer

Traditional, planar slicers have been required to slice in a plane-by-plane fashion because upper layers require the support of the lower layers to remain in their intended position. With the advent of embedded printing (Bhattacharjee et al., “Writing in the Granular Gel Medium,” Science Advances 1(8):4-10 (2015); Hinton et al., “Three-dimensional Printing of Complex Biological Structures by Freeform Reversible Embedding of Suspended Hydrogels,” Science Advances 1(9) (2015), which are hereby incorporated by reference in their entirety), the printed material does not necessarily require that layers below support upper layers because the support bath material maintains the structure, rather than the printed material itself. Novel slicers have been developed to enable highly controlled print path generation (Gleadall, “FullControl GCode Designer: Open-source Software for Unconstrained Design in Additive Manufacturing,” Additive Manufacturing 46:102109 (2021), which is hereby incorporated by reference in its entirety); however, no slicer has been developed to take advantage of the flexibility in print direction that embedded printing allows. The BENT slicer described herein has been developed with the intention of creating 3D printer-ready files to allow for flexibility in the Z-axis movement of printing, supported by embedded printing methods.

The BENT slicer described herein can replicate CAD surface models with continuous print paths that follow non-planar travel. The slicer can handle continuous and discontinuous surfaces if no point occurs directly above another on the initial surface geometry. The slicer can create paths on either one axis or on two perpendicular axes to create a cross-hatching pattern between alternating layers. 3D surfaces are created by duplicating layers to the desired print thickness.

Because this version of the slicer divides the model into equally spaced sections in the X-Z and Y-Z planes, the effective spacing between parallel paths may not be equal depending on the surface angle. On a sloped surface, the path spacing becomes the hypotenuse of the rise and run, increasing the distance between adjacent paths. Adjusting the path spacing based on angle is an alternative feature that may improve consistency of material deposition no matter the surface angle.

### Effect of Extruder Direction on Alginate Extrusion Width

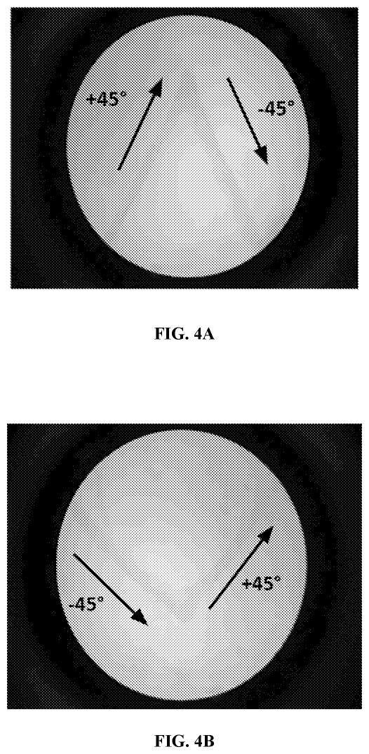

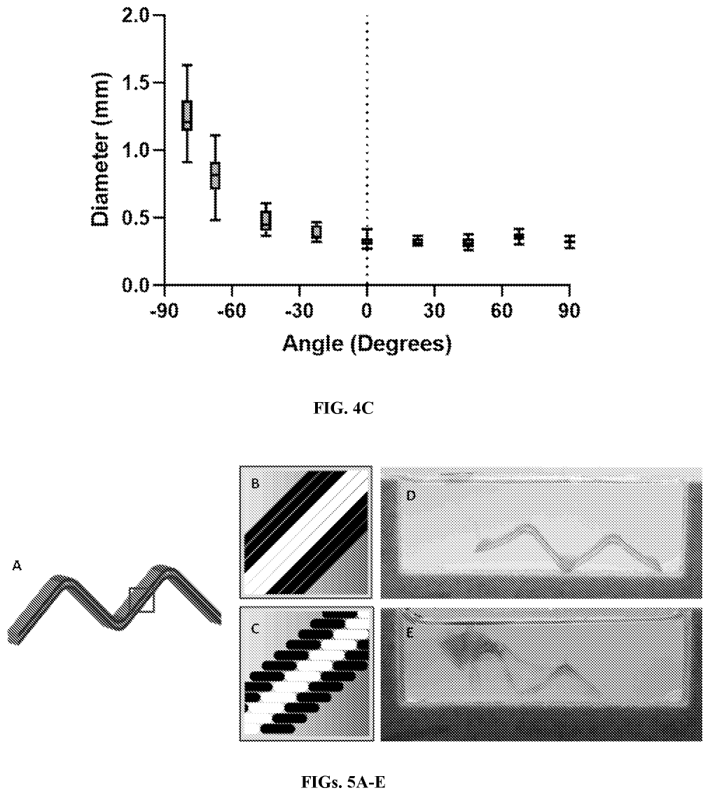

Bright-field microscopy was used to measure extrusion width of alginate extruded in multiple directions. As shown in FIGS. 4A-2C, measurement of path width with respect to printed angle demonstrates that upward motion leads to no significant difference in path width compared to horizontal movement. Downward movements of 45 degrees and greater display extrusion widths greater than the nozzle diameter. The steeper the downward angle, the greater the diameter and the more variable the measurements. This is likely due to the needle passing through or near the bioink as it is extruded, smearing the line. These results demonstrate that print geometries may be limited in their maximum feature angles because of the limitations of downward print angles. Changes in the nozzle shape and size may impact the resulting path by changing the dynamic interaction of the nozzle, ink, and bath material. Future consideration of limiting downwards print paths may be a key to high fidelity printing results. The use of complex print mechanics such as six-axis printing could prevent this limitation by maintaining the print nozzle normal to the direction of movement so that all print paths should maintain the same extrusion features as a horizontal print using three-axis methods.

Future work in simulating the fluid flow will help to determine factors that improve the consistency of print path diameter. Factors such as nozzle shape and size will likely affect the rheology of the extruded material through the bath. Additional studies of the behavior of other printable biomaterials, such as collagen or methacrylated hyaluronic acid, will also develop a better understanding of the process based on crosslinking/gelation process and rheology.

Multi-Material Printing with BENT and Planar Slicing

It has been demonstrated that, with the BENT method, multi-material geometries can be printed with distinctly different results than traditional slicing methods (FIGS. 5A-E). The BENT method follows the curvature of the model in order to maintain the fidelity of the final print while the planar sliced model builds up the geometry in slices of the Z-axis (FIGS. 5B-C). Because there are two separate materials printed, the printer must switch between extruders to complete each particular layer (FIG. 5E). At every material change, the extruder must move completely out of the print bath and be replaced by the other nozzle. Any excess material coming out of these nozzles during the transition may get deposited at the point of entry and exit into the bath. This accumulation of material is apparent in the top left of the planar print (FIG. 5E). Planar printing also requires travel moves between parts of the structure that are at the same height in the Z-axis. The extruders do not deposit during these moves, but the motion can drag deposited material and any excess oozing can cause stringing between the two points of deposition. This effect can be seen between the peaks of the planar print (FIG. 5E).

The BENT print extruders travel along the curvature of the model, depositing material without any travel moves within a layer (FIG. 5D). There are only two extruder changes during the print when switching from black to clear and then back to black. This leads to very little material accumulation due to movement in and out of the container. There is a very distinct differentiation between the three layers, except at the lowest center point of the print. The excessive blending in the valley of the print may be due to material being pushed to low points when the nozzle is moving at a downward angle.

### Mechanics of Corrugated Alginate Prints

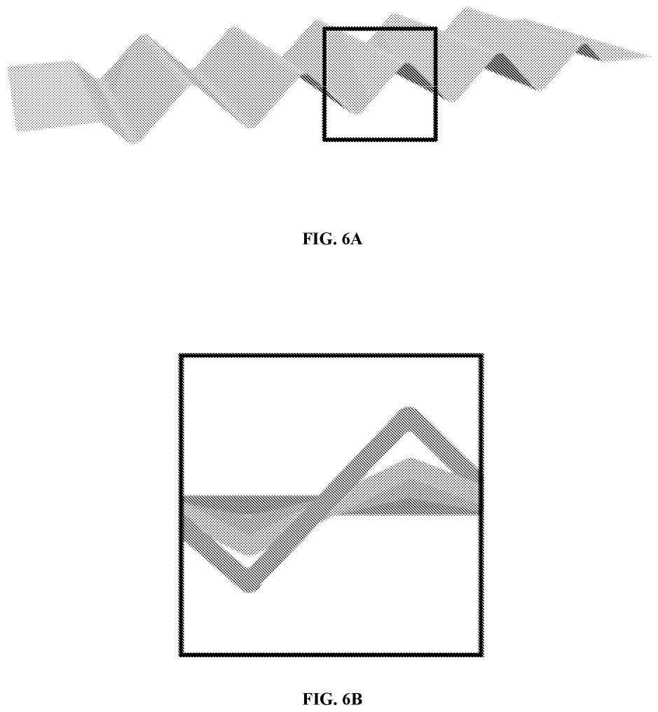

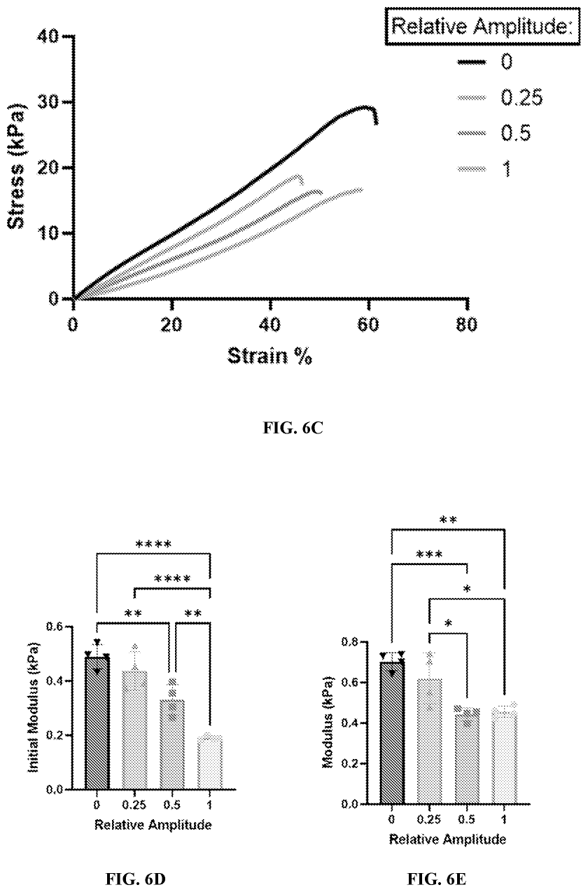

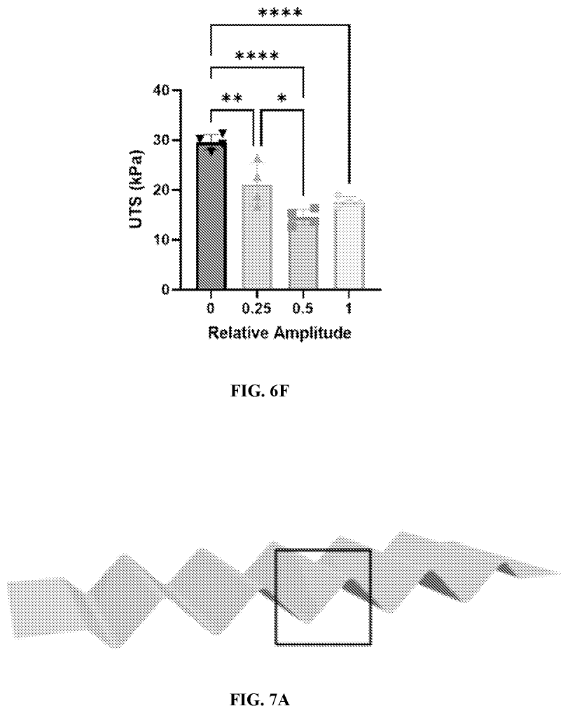

Corrugated geometries created out of soft, flexible materials such as hydrogels allow for unfolding of the angular portions up to a certain strain. Simple corrugations were created, as shown in FIG. 6A, using the non-planar printing method to understand how to create geometries that alter the stress-strain relationship to be tunable towards properties of native tissues (Mehta et al., “Engineering Biologically Extensible Hydrogels Using Photolithographic Printing,” Acta Biomaterialia 75:52-62 (2018), which is hereby incorporated by reference in its entirety).

Amplitude of the corrugation was altered from flat prints to 45 degree angle corrugations with 0.5 mm fillet diameter as shown in FIG. 6B. After printing, the prints were stretched to failure to quantify the mechanical properties (FIG. 6C). Increasing the amplitude of the prints lowers the initial modulus of the prints (FIG. 6D). This is likely due to greater folding length that takes more strain to reach the point of flattening out. Corrugations also trend towards upward J-shaped curves. The maximum modulus of the flat samples is greater than that of the corrugated samples, with the trend decreasing with greater amplitude (FIG. 6E). Corrugated samples likely have high stress concentrations at the peaks and valleys of the print that may not be able to fully straighten out before failure. A similar trend is seen in FIG. 6F with the ultimate tensile stress, strengthening the idea that the corrugations increase stress concentrations at the peaks and valleys, leading to premature failure compared to flat samples.

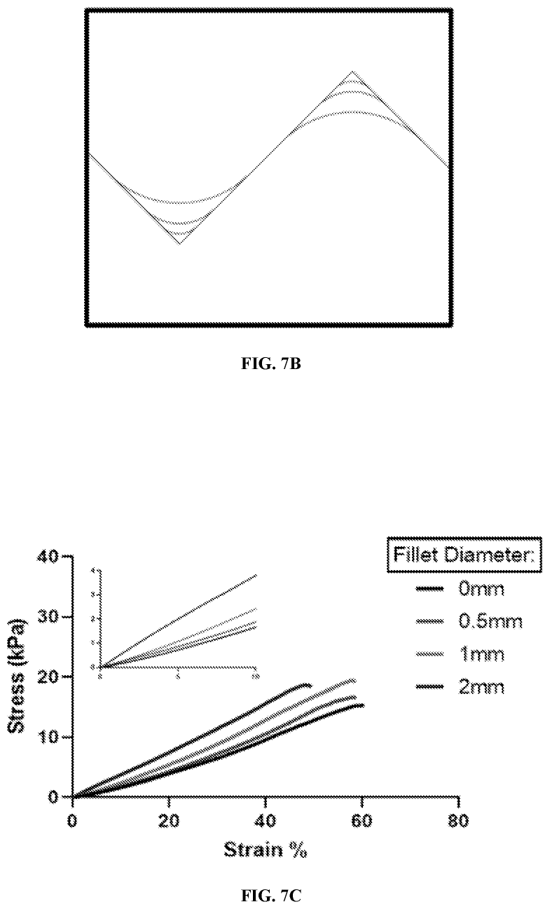

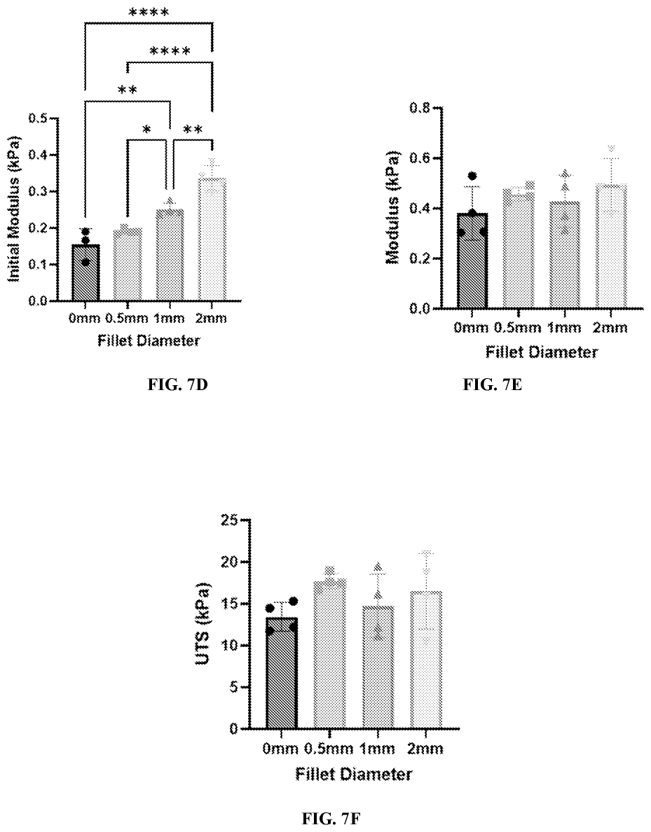

Fillet diameter was similarly altered in the base corrugated geometry (FIG. 7A) to understand how the folds further contribute to the mechanical response to stretch. The fillet diameter ranged from 0 mm (90 degree intersection) to 2 mm (FIG. 7B). With stretch to failure, it was found that increasing the fillet diameter creates a higher initial modulus and reduced strain stiffening as shown in FIGS. 7C-D. The higher the fillet diameter, the less straight sections of hydrogel exist in the geometry, leading to less of a hinge-like motion at the peaks and valleys and more of a constant, linear, spring-like response to strain. The maximum modulus and ultimate tensile stress show no significant trends with respect to the fillet diameter, showing that the stress concentrations are likely similar across these samples (FIGS. 7E-F).

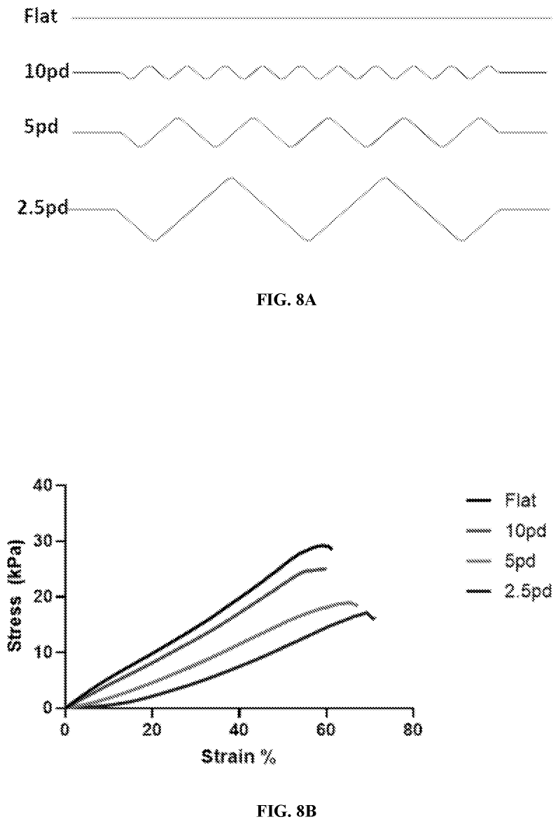

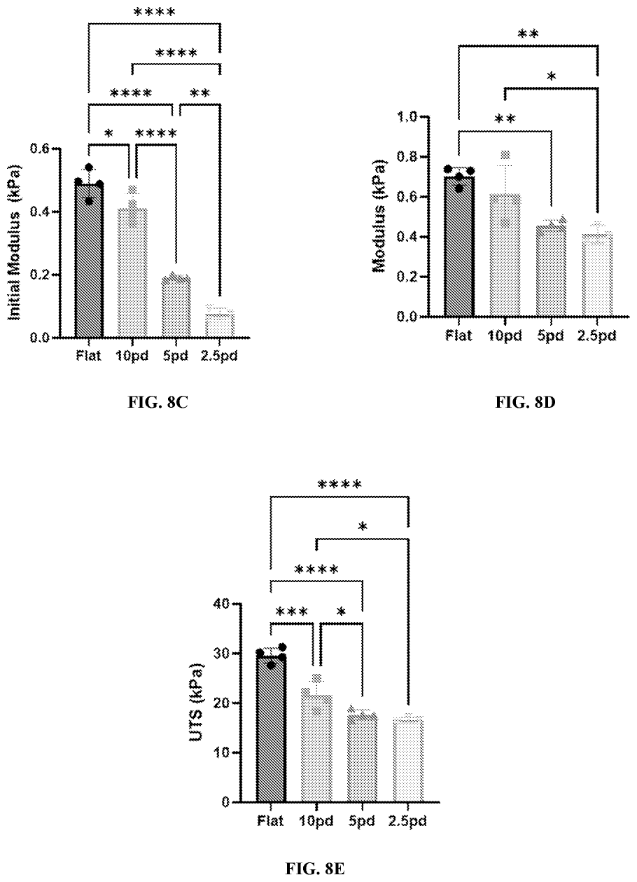

Lastly, several variations were created on the base corrugation by altering the number of periods contained within the same sample length. The intersections were kept at 45 degrees for all samples, leading to high amplitude in low periodicity samples and vice versa (FIG. 8A). Lower periodicity created greater strain stiffening and significantly lower initial modulus (FIGS. 8B-C). However, FIGS. 8D-E show lower periodicity also lowers maximum modulus and ultimate tensile strength. Because there are fewer hinge points, the stress of unfolding is distributed to fewer points, leading to failure at a lower stress.

These studies of corrugated geometries show that strain-stiffening materials can be created out of a homogeneous alginate bioink. However, balancing the desired stress-strain properties, the failure stress, and the maximum modulus needs to be considered depending on the application needs.

### Effect of Path Direction on Response to Stretch

FRESH bioprinting gradually crosslinks bioinks through aqueous phase of the bath material. In the case of printing alginate, calcium chloride in the print bath diffuses into the bioink to covalently crosslink the alginate (Hinton et al., “Three-dimensional Printing of Complex Biological Structures by Freeform Reversible Embedding of Suspended Hydrogels,” Science Advances 1(9) (2015), which is hereby incorporated by reference in its entirety). This process is slow enough that adjacent print paths can fuse to one another, but fast enough that paths still contain distinct directionality after washing. It was sought to demonstrate how this new complex feature effects the anisotropy of printed samples.

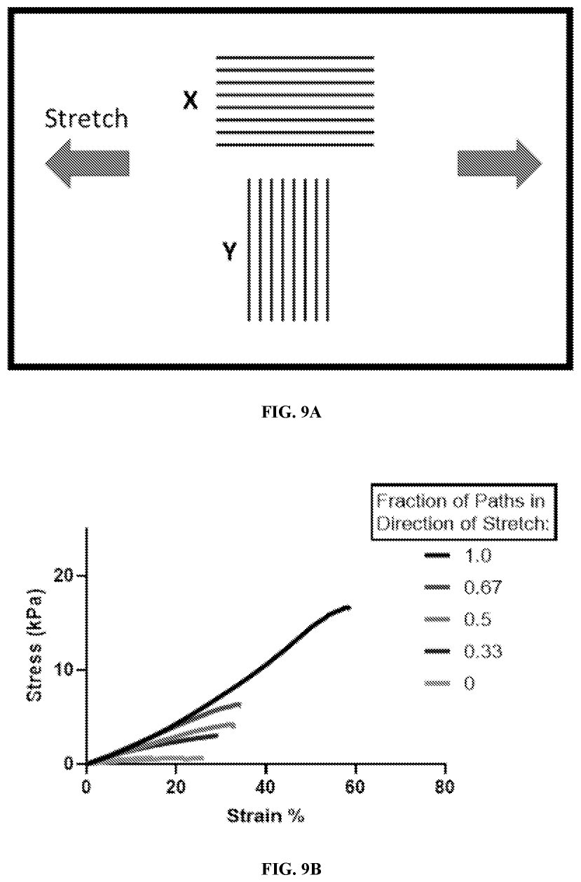

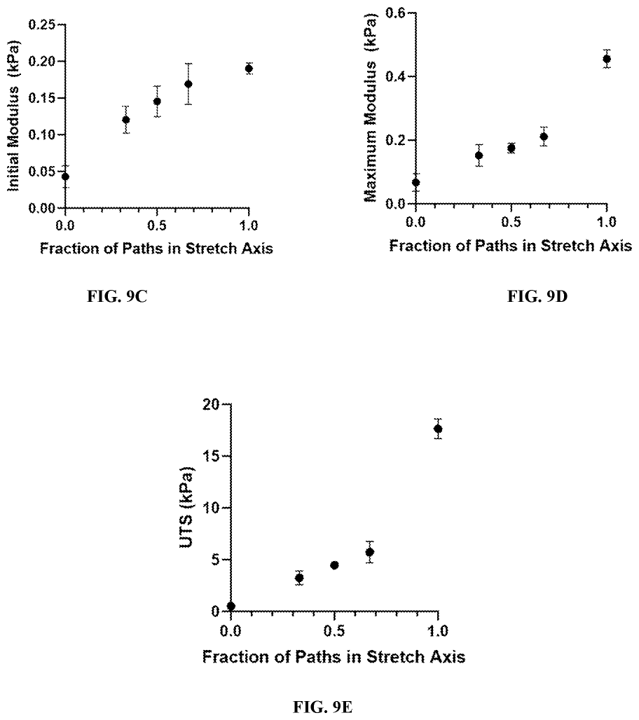

The same base corrugation was printed as previously described. However, path direction was modified depending on the sample. Direction ranged from all six paths perpendicular to the axis of stretch to all six paths parallel to the axis of stretch (FIG. 9A). Samples in between were printed with alternating layers of different directions. Stretch to failure revealed that when all six layers are aligned with the stretch, the failure strain and stress are higher than any sample with layer(s) perpendicular (FIG. 9B). The more perpendicular paths that are included, the lower the initial modulus, maximum modulus, and ultimate tensile stress (FIGS. 9C-E). These features are likely due to delamination of layers perpendicular to the stress. This anisotropy depending on path direction may be useful for biaxial strengthening of tissues that will be loaded in multiple directions (Bian et al., “Controlling the Structural and Functional Anisotropy of Engineered Cardiac Tissues,” Biofabrication 6(2) (2014); Dwyer & Coulombe, “Cardiac Mechanostructure: Using Mechanics and Anisotropy as Inspiration for Developing Epicardial Therapies in Treating Myocardial Infarction,” Bioactive Materials 6(7):2198-2220 (2021), which are hereby incorporated by reference in their entirety), or creating materials with highly complex “bulk” responses in various material directions and positions without the need for unique biomaterial formulations for each location. Much research in polymer chemistry has encountered significant challenges in creating nonlinear elastic materials that mimic biological materials properties. This is likely because those polymers are homogeneous and isotropic, whereas native tissues exhibit positionally varying anisotropic microstructure of multiple simpler materials to achieve more complex material properties. This is the simplicity and elegance of the manufacturing approach of the present disclosure, which unlocks previously unattainable bulk property complexity through the complex arrangement of simpler materials.

### Printing Miura Ori Origami Structures

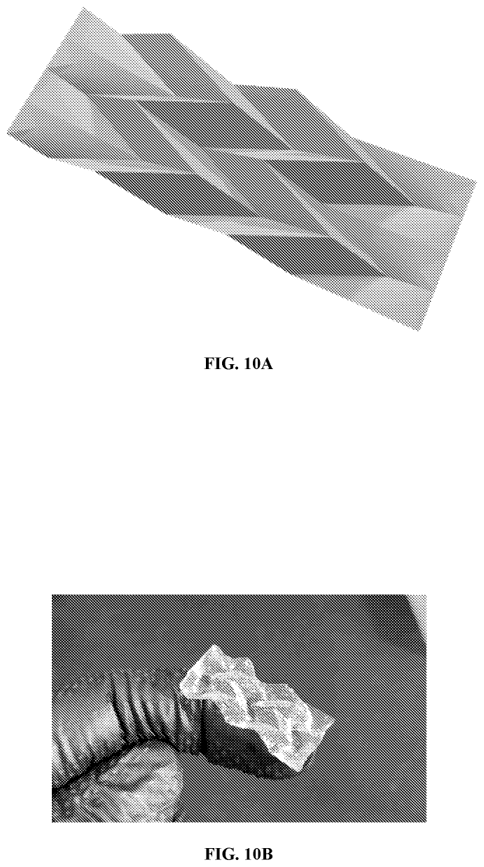

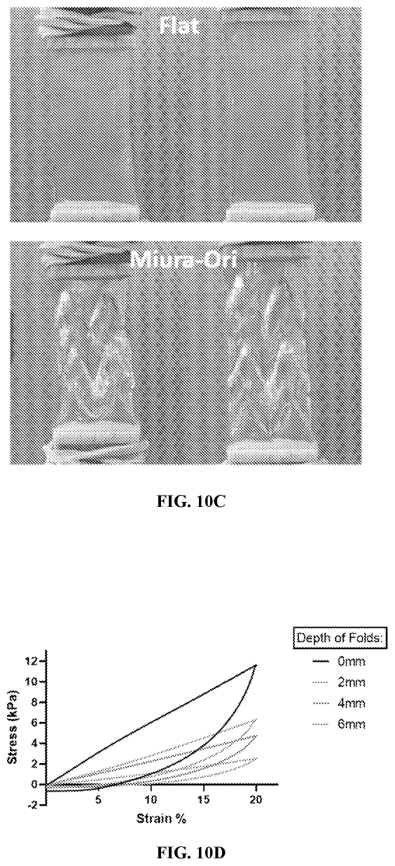

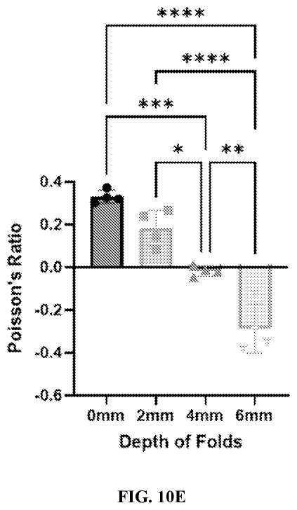

Previous studies have demonstrated applications of auxetic materials in tissue engineering (Jin et al., “Fabrication of Multi-scale and Tunable Auxetic Scaffolds for Tissue Engineering,” Materials and Design 197:109277 (2021); Mardling et al., “The Use of Auxetic Materials in Tissue Engineering,” Biomaterials Science 8(8):2074-2083 (2020), which are hereby incorporated by reference in their entirety). These materials have been auxetic because of open spaces in the geometry that allowed for in-plane flexure of the material. As shown in FIG. 10A, the Miura-Ori geometry is a continuous surface geometry that presents auxetic properties by unfolding, making it optimal for applications where a continuous, solid surface is desired (Silverberg et al., “Using Origami Design Principles to Fold Reprogrammable Mechanical Metamaterials,” Science 345(6197):647-650 (2014), which is hereby incorporated by reference in its entirety). The Miura-Ori geometry printed with alginate displays the desired surface features of the original geometry (FIG. 10B). When stretched, the unfolding of the structure creates a change in width that is directly related to the depth of the folds (FIG. 10C). The stress response during cyclic stretch decreases with the inclusion of these origami folds, and the deeper the folds are, the lower the stress (FIG. 10D). This is similar to the trend found from changing the amplitude of corrugated prints. The increased feature size creates greater surface area in the same amount of space, allowing for longer periods of unfolding before the sample flattens out. Measurement of the Poisson's ratio during stretch to 20% strain revealed significant reduction in the ratio of the Miura-Ori prints (FIG. 10E). 4 mm depth of fold prints have a ratio near zero, meaning that the width of the sample remains constant within this range of stretch. 6 mm depth of fold prints have a negative Poisson's ratio, meaning that the prints become wider during stretch. These features may be able to reduce off-axis stress if the entire print is constrained and uniaxially stretched. This may be useful for patch-like biomaterial applications where there is risk of detachment on certain edges of the tissue (sources).

### CONCLUSIONS

Manufacturing articles for applications in tissue engineering requires material geometry and mechanical properties to closely match those of the native tissue it replaces. To address this, a method of flexible manufacturing for creating curvature in embedded printing was developed. It is shown that this method can recreate thin, curved geometries with single and dual material printing. It is also shown that the process allows for production of non-linear stress-strain relationships within a material that may create a more native-like response to stress. Additionally, anisotropic behavior through layer alignment creates a secondary method of property development. These, combined with other complex behaviors, such as negative Poisson's ratio geometry, create an array of techniques in non-planar printing that improve the ability to fabricate native mechanical properties.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A method of fabricating an article, said method comprising:
obtaining a non-planar surface model of an article to be fabricated;
creating X and Y coordinates of the surface to create an X-Y plane;
creating planes orthogonal to the X-Y plane to obtain lines of intersection between the surface and said planes;
correlating coordinates of intersecting lines to create non-planar print paths; and
printing along the print paths to fabricate the article.

2. The method of claim 1, wherein the X-Y plane comprises X-Z and Y-Z planes.

3. The method of claim 1 or claim 2 further comprising:
offsetting the non-planar print paths to create alternating print paths to a desired thickness of the article.

4. The method of any one of the preceding claims further comprising:
calculating printing distance of a print head to a print substrate based on the distance between sequential points on the print paths.

5. The method of any one of the preceding claims further comprising:
converting points that comprise the print paths to strings; and
combining the strings with extrusion values to create lines of a GCODE file.

6. The method of any one of the preceding claims, wherein said printing is carried out by extrusion of a solution from a print head.

7. The method of claim 6, wherein the print head is moveable in X, Y, and Z directions.

8. The method of claim 6 or claim 7, wherein said printing is carried out with multiple solutions from a single nozzle of the print head.

9. The method of any one of claims 6-8, wherein said printing is carried out with multiple solutions from multiple nozzles from said print head or multiple print heads.

10. The method of any one of claims 6-9, wherein the solution comprises living cells.

11. The method of claim 10, wherein the article is a living tissue.

12. The method of any one of the preceding claims, wherein said printing is carried out in a support bath.

13. The method of claim 12, wherein the support bath comprises FRESH.

14. The method of any one of the preceding claims, wherein said printing comprises printing layers of different thickness.

15. The method of any one of the preceding claims, wherein said print paths comprise curvilinear print paths.
